(19) United States
(12) Patent Application Publication
(10) Pub. No.: US 2010/0021379 A1
(43) Pub. Date: Jan. 28, 2010

(54) Chemical Antibodies for Immunotherapy and Imaging

(75) Inventors: Kit S. Lam, Davis, CA (US); Pappanaicken R. Kumaresan, Sacramento, CA (US); Amanda Enstrom, Sacramento, CA (US); Ruiwu Liu, Sacramento, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/306,244

(22) PCT Filed: Jun. 28, 2007
(86) PCT No.: PCT/US07/72400

§ 371 Date: Jul. 27, 2009

Related U.S. Application Data
(60) Provisional application No. 60/817,667, filed on Jun. 29, 2006.

(51) Int. Cl. A61K 51/00; A61K 49/00; A61K 39/395; C12Q 1/02; C12N 9/96; C07K 16/00

(52) U.S. Cl. 424/1.49; 424/9.1; 424/9.6; 424/178.1; 435/29; 435/188; 530/391.1; 530/391.3; 530/391.5; 530/391.7; 530/391.9

(57) ABSTRACT

The present invention provides antibody conjugates comprising a targeting agent covalently attached to an antibody or fragment thereof. The antibody conjugates of the present invention are particularly useful for imaging a tumor, organ, or tissue and for treating diseases and disorders such as cancer, inflammatory diseases, autoimmune diseases, infectious diseases, and neurological disorders. Kits containing the antibody conjugates described herein find utility in a wide range of applications including, for example, in vivo imaging and immunotherapy.

### BACKGROUND OF THE INVENTION

Antibodies are highly specific, naturally evolved molecules that recognize and eliminate pathogenic and disease antigens. Cancer, inflammatory diseases, and autoimmune diseases are an important focus of antibody therapy (Brekke et al., Natural Reviews Drug Discovery, 2:52-62 (2003)). For example, antibodies have been developed to bind specific cytokines or their receptors. Inhibition of cytokines associated with inflammation and the modulation of the immune response by immune cell depletion has been shown to be a viable therapy for autoimmune diseases. The list of approved antibody therapeutics against cancer, viral diseases, and inflammatory diseases is growing rapidly (Carter, Natural Review Cancer, 1:118-129 (2001); Hudson et al., Expert Opinion in Biology and Therapeutics, 1:845-855 (2001)).

Engineered antibodies developed by recombinant technology revolutionized antibody usage to fight against various pathological and infectious diseases (Hudson et al., Nature Medicine, 9:129-134 (2003)). Intact antibody therapy has been more successful against circulating cancer cells than solid tumors because of the greater accessibility of lymphoma and leukemia cells to intact monoclonal antibodies (mAbs). However, most antibodies penetrate very slowly and in a non-uniform fashion in solid tumors. In addition, high levels of these antibodies in serum induce Fc-receptor associated toxicities (Jain, Cancer Research, 47:3039-3051 (1987)).

Recent pharmacokinetic and biodistribution studies indicate that intermediate-sized multivalent molecules such as diabodies (˜55 kDa) provide rapid tissue penetration, high target retention, and rapid blood clearance (Kenanova et al., Cancer Research, 65:622-631 (2005); Robinson et al., Cancer Research, 65:1471-1478 (2005)). To reduce the size of the antibody, most studies have been devoted to modifying the antigen binding portion of the human IgG to monobodies (single chain Fvs, ˜28 kDa), bis-scFV (bispecific, ˜55 kDa), diabodies (bispecific, ˜50 kDa), triabodies (trivalent, ˜75 kDa), or tetrabodies (tetravalent, ˜100 kDa). However, small scFV fragments (˜30 kDa) are cleared extremely rapidly and have poor tumor retention because of their monovalent binding properties (Tominson et al., Methods of Enzymology, 326:461-479 (2000); Todorovska et al., J. Immunol. Methods, 248:47-66 (2001)).

Recent biodistribution studies have demonstrated that diabodies, because of their size, are rapidly eliminated through kidneys, thereby limiting the exposure to bone marrow, which is most often the dose limiting organ with intact radiolabled mAbs (Sundaresan et al., J. of Nuclear Medicine, 44:1962-1969 (2003)). Diabodies are generally dimeric molecules comprising polypeptides with a heavy chain variable domain connected to a light chain variable domain. However, diabodies lack the antibody Fc region and are thus unable to generate an immune response by recruiting immune effector cells and activating complement proteins. As a result, diabodies do not kill cancer cells and can only be used to deliver therapeutic payloads such as radionuclides, drugs, and enzymes to the tumor.

Larger bivalent molecules such as minibodies (scFv-C_{H}3 dimers) and scFv₂-Fc can accumulate at a higher abundance in tumors and can be designed with a spectrum of serum half-lives by modulating the interaction with FcRn receptors (Woof, et al., Nature Reviews and Immunology, 4:89-99 (2004)). Minibodies are capable of achieving a higher total tumor uptake, substantially faster clearance, and better tumor-to-blood ratios than either intact immunoglobulin (150 kDa) or Fab′₂(110 kDa) (Olafsen et al., Protein Engineering Designing and Selection, 17:315-323 (2004)). However, like diabodies, minibodies are also deficient in recruiting host immune effector cells and activating the host complement system.

In view of the foregoing, there is a need in the art for tumor targeting agents which not only provide rapid tumor penetration, high tumor retention, and rapid blood clearance, but are also capable of generating an appropriate immune response by modulating immune cell activity. The present invention satisfies this and other needs.

### BRIEF SUMMARY OF THE INVENTION

The present invention provides antibody conjugates comprising a targeting agent covalently attached to an antibody or fragment thereof. The antibody conjugates of the present invention are particularly useful for imaging a tumor, organ, or tissue and for treating diseases and disorders such as cancer, inflammatory diseases, autoimmune diseases, infectious diseases, and neurological disorders. Kits containing the antibody conjugates described herein find utility in a wide range of applications including, for example, in vivo imaging and immunotherapy.

In one aspect, the present invention provides a conjugate comprising:
(a) an antibody having at least a portion of a constant region; and
(b) a targeting agent comprising a functional group covalently attached to a complementary functional group present in the constant region.

The antibody component of the conjugate can comprise any immunoglobulin class (e.g., IgG, IgM, IgA, IgD, or IgE) or isotypes thereof (e.g., IgG1, IgG2, IgG3, or IgG4). In some embodiments, the portion of the constant region comprises at least two heavy chain constant domains. Examples of heavy chain constant domains include, but are not limited to, CH1, CH2, CH3, and/or CH4 constant domains. In other embodiments, the portion of the constant region further comprises a hinge region.

In certain embodiments, the antibody component of the conjugate comprises an antibody fragment. As a non-limiting example, the antibody can comprise a dimer of heavy chain fragments in which each heavy chain fragment independently comprises at least two heavy chain constant domains. In particular, each heavy chain fragment can comprise CH1, CH2, and CH3 constant domains, CH2 and CH3 constant domains, or CH1 and CH2 constant domains. In some instances, each heavy chain fragment can further comprise a hinge region. In other instances, each heavy chain fragment does not comprise a variable domain. Preferably, the dimer of heavy chain fragments comprises an Fc fragment, wherein each heavy chain fragment comprises CH2 and CH3 constant domains and a hinge region.

The antibody conjugates of the present invention typically have a molecular weight of at least about 25 kDa, e.g., at least about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 kDa. For example, an antibody conjugate comprising a whole antibody molecule (e.g., whole IgG1) can have a molecular weight of at least about 150 kDa. With regard to antibody fragments, an antibody conjugate in which each heavy chain fragment comprises CH1, CH2, and CH3 constant domains can have a molecular weight of from about 90 to about 110 kDa, whereas an antibody conjugate in which each heavy chain fragment comprises CH2 and CH3 constant domains or CH1 and CH2 constant domains can have a molecular weight of from about 50 to about 60 kDa.

In some embodiments, the targeting agent comprises a ligand such as, e.g., a small organic molecule, peptide, peptidomimetic, peptoid, protein, polypeptide, glycoprotein, oligosaccharide, or nucleic acid. For example, the targeting agent can comprise a ligand (e.g., a peptide or peptidomimetic) specific for cell-surface receptors integrin receptors, lectin receptors, immunoglobulin receptors, receptor tyrosine kinases, growth factor receptors, insulin receptors, fibroblast growth factor receptors, neurotrophin receptors, Eph receptors, G-protein coupled receptors, adrenergic receptors, olfactory receptors, NMDA receptors, Toll-like receptors, T cell receptors, and the like. Additional ligands useful as targeting agents include, but are not limited to, peptide or peptidomimetic ligands which bind to a target protein on a pathogen, microbial agent, or infectious agent (e.g., a virus, bacterium, fungus, parasite, or any other infectious agent), peptide or peptidomimetic ligands which bind to a target protein aggregate such as amyloid, prions, and the like, peptide or peptidomimetic ligands which bind to a toxin or metabolite derived from host metabolism or from a pathogen, microbial agent, or infectious agent, and peptide or peptidomimetic ligands which bind to a poisonous agent such as snake venom or drugs.

Preferably, the targeting agent comprises a ligand (e.g., a peptide or peptidomimetic ligand) specific for an integrin receptor expressed by a particular cell, tumor, tissue, or organ. The integrin receptor can be, e.g., α₄β₁, α₅β₁, αᵥβ₃, or αᵥβ₅ integrin. A non-limiting example of a peptidomimetic ligand specific for α₄β₁ integrin that is suitable for use in the antibody conjugates of the present invention has the following formula:

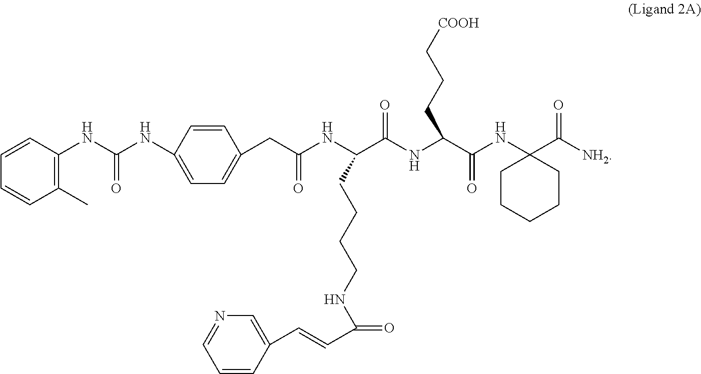

In some embodiments, the functional group on the targeting agent comprises a maleimide group and the complementary functional group present in the constant region of the antibody or fragment thereof comprises a thiol group. In other embodiments, the functional group on the targeting agent comprises an oxidized 3,4-dihydroxy-phenylalanine (DOPA) group and the complementary functional group present in the constant region of the antibody or fragment thereof comprises an amine group. In further embodiments, the functional group on the targeting agent comprises an aminoxy group and the complementary functional group present in the constant region of the antibody or fragment thereof comprises a ketone group (e.g., methyl-ketone group). In certain instances, the ketone group is present on a linker such as, for example, a heterobifunctional linker attached to an amine group (e.g., ε-amino group of lysine) present in the constant region. Additional examples of functional groups suitable for the non-site specific covalent attachment of a targeting agent to an antibody or fragment thereof are shown in Table 1, wherein the bond results from a reaction between complementary functional groups present on the two molecular species. In a preferred embodiment, the complementary functional group is not present at the amino-terminus or carboxyl-terminus of the antibody or fragment thereof, i.e., the complementary functional group is present at an internal site in the antibody molecule.

In other embodiments, the antibody further comprises a carboxyl-terminal polyarginine linker. In certain instances, the polyarginine linker further comprises a therapeutic agent (e.g., radionuclide, toxin, etc.) and/or an imaging agent (e.g., radionuclide, biotin, a fluorophore, a fluorescent protein, horseradish peroxidase, alkaline phosphatase, etc.) attached thereto. In some instances, a radionuclide that is being used as a therapeutic agent or an imaging agent can be bound to a chelating agent.

In another aspect, the present invention provides a method for treating a disease or disorder in a subject in need thereof, the method comprising:
administering to the subject a therapeutically effective amount of a conjugate comprising an antibody having at least a portion of a constant region and a targeting agent comprising a functional group covalently attached to a complementary functional group present in the constant region.

The antibody conjugates described herein are suitable for treating a wide variety of diseases and disorders, e.g., in human or veterinary subjects. Examples include, but are not limited to, cancer, an inflammatory disease, an autoimmune disease, an infectious disease, and a neurological or musculoskeletal disorder.

In some embodiments, the cancer comprises renal cell carcinoma, bladder cancer, prostate cancer, testicular cancer, ovarian cancer, cervical cancer, lung cancer, breast cancer, colon cancer, stomach cancer, head and neck cancer, brain cancer, bone cancer, hepatocarcinoma, leukemia, lymphoma, or multiple myeloma.

In other embodiments, the inflammatory disease comprises inflammatory bowel disease, a rheumatoid disease, fibrositis, pelvic inflammatory disease, acne, psoriasis, actinomycosis, dysentery, biliary cirrhosis, Lyme disease, heat rash, Stevens-Johnson syndrome, mumps, pemphigus vulgaris, or blastomycosis.

In yet other embodiments, the autoimmune disease comprises Type I diabetes mellitus, myasthenia gravis, vitiligo, Graves' disease, Hashimoto's disease, Addison's disease, autoimmune gastritis, autoimmune hepatitis, systemic lupus erythematosus, progressive systemic sclerosis and variants, polymyositis, dermatomyositis, pernicious anemia, primary biliary cirrhosis, autoimmune thrombocytopenia, Sjögren's syndrome, or multiple sclerosis.

In additional embodiments, the infectious disease comprises AIDS/HIV or HIV-related disorders, Alpers syndrome, anthrax, bovine spongiform encephalopathy, chicken pox, cholera, conjunctivitis, Creutzfeldt-Jakob disease, dengue fever, Ebola, elephantiasis, encephalitis, fatal familial insomnia, Fifth's disease, Gerstmann-Straussler-Scheinker syndrome, hantavirus, helicobacter pylori, hepatitis (hepatitis A-C), herpes, influenza (e.g., avian influenza A), Kuru, leprosy, lyme disease, malaria, hemorrhagic fever (e.g., Rift Valley fever, Crimean-Congo hemorrhagic fever, Lassa fever, Marburg virus disease, and Ebola hemorrhagic fever), measles, viral or bacterial meningitis, mononucleosis, nosocomial infections, otitis media, pelvic inflammatory disease, plague, pneumonia, polio, prion disease, rabies, rheumatic fever, roseola, Ross River virus infection, rubella, salmonellosis, septic arthritis, sexually transmitted diseases, shingles, smallpox, strep throat, tetanus, toxic shock syndrome, toxoplasmosis, trachoma, tuberculosis, tularemia, typhoid fever, valley fever, whooping cough, or yellow fever.

In further embodiments, the neurological or musculoskeletal disorder comprises Alzheimer's disease, Aicardi syndrome, amnesia, amyotrophic lateral sclerosis, anencephaly, aphasia, arachnoiditis, Arnold Chiari malformation, ataxia telangiectasia, Batten disease, Bell's palsy, brachial plexus injury, brain injury, brain tumor, Charcol-Marie-Tooth disease, encephalitis, epilepsy, essential tremor, Guillain-Barre Syndrome, hydrocephalus, hyperhidrosis, Krabbes disease, meningitis, Moebius syndrome, muscular dystrophy, multiple sclerosis, Parkinson's disease, peripheral neuropathy, postural or orthostatic tachycardia syndrome, progressive supranuclear palsy, Reye's syndrome, shingles, Shy-Drager Syndrome, spasmodic torticollis, spina bifida, spinal muscular atrophy, Stiff Man syndrome, synesthesia, syringomyelia, thoracic outlet syndrome, Tourette syndrome, toxoplasmosis, or trigeminal neuralgia.

As described above, the antibody component of the conjugate can comprise any immunoglobulin class such as, e.g., IgG, IgM, IgA, IgD, IgE, or isotypes thereof. The portion of the constant region generally comprises at least two heavy chain constant domains. Non-limiting examples of heavy chain constant domains include CH1, CH2, CH3, and/or CH4 constant domains. In certain embodiments, the portion of the constant region further comprises a hinge region.

In some embodiments, the antibody component of the conjugate comprises an antibody fragment such as, e.g., a dimer of heavy chain fragments in which each heavy chain fragment independently comprises at least two heavy chain constant domains. For example, each heavy chain fragment can comprise CH1, CH2, and CH3 constant domains, CH2 and CH3 constant domains, or CH1 and CH2 constant domains. In some instances, each heavy chain fragment can further comprise a hinge region. In other instances, each heavy chain fragment does not comprise a variable domain. In a preferred embodiment, the dimer of heavy chain fragments comprises an Fc fragment, wherein each heavy chain fragment comprises CH2 and CH3 constant domains and a hinge region.

In other embodiments, the targeting agent comprises a ligand (e.g., a peptide or peptidomimetic) specific for a cell-surface receptor such as an integrin receptor (e.g., α₄β₁ integrin). As a non-limiting example, a chemical ligand such as the peptidomimetic ligand specific for α₄β₁ integrin shown above (i.e., Ligand 2A) can be linked to an antibody or fragment thereof (e.g., Fc fragment) via non-site specific conjugation to form an antibody conjugate that is suitable for use in treating a disease or disorder in accordance with the present invention. The conjugation can occur, for example, by means of a maleimide functional group present on the targeting agent and a complementary thiol functional group present in the constant region of the antibody or fragment thereof. In certain other instances, the conjugation can occur by means of an oxidized DOPA functional group present on the targeting agent and a complementary amine functional group present in the constant region of the antibody or fragment thereof. Alternatively, the conjugation can occur by means of an aminoxy functional group present on the targeting agent and a complementary ketone functional group present in the constant region of the antibody or fragment thereof. Additional examples of complementary functional groups suitable for the non-site specific covalent attachment of a targeting agent to an antibody or fragment thereof are shown in Table 1. Preferably, the complementary functional group is not present at the amino-terminus or carboxyl-terminus of the antibody or fragment thereof, i.e., the complementary functional group used for the covalent attachment of a targeting agent is present at an internal site in the antibody molecule.

In yet another aspect, the present invention provides a method for imaging a tumor, organ, or tissue, the method comprising:
(a) administering to a subject in need of such imaging, a conjugate comprising an antibody having at least a portion of a constant region and a targeting agent comprising a functional group covalently attached to a complementary functional group present in the constant region; and
(b) detecting the conjugate to determine where the conjugate is concentrated in the subject.

In certain embodiments, the antibody component of the conjugate can comprise any immunoglobulin class (e.g., IgG, IgM, IgA, IgD, or IgE) or isotypes thereof. The portion of the constant region on each heavy chain of the antibody molecule typically comprises at least two heavy chain constant domains (e.g., CH1, CH2, CH3, and/or CH4 constant domains) and can further comprise a hinge region.

In other embodiments, the antibody component of the conjugate comprises an antibody fragment. For example, the antibody can comprise a dimer of heavy chain fragments in which each heavy chain fragment independently comprises at least two heavy chain constant domains. In certain instances, the dimer of heavy chain fragments comprises a molecule wherein each heavy chain fragment comprises CH1, CH2, and CH3 constant domains, CH2 and CH3 constant domains, or CH1 and CH2 constant domains. In some instances, each heavy chain fragment can further comprise a hinge region. In other instances, each heavy chain fragment does not comprise a variable domain. Preferably, the dimer of heavy chain fragments comprises an Fc fragment, wherein each heavy chain fragment comprises CH2 and CH3 constant domains and a hinge region.

In further embodiments, the targeting agent comprises a ligand (e.g., a peptide or peptidomimetic) specific for a cell-surface receptor such as an integrin receptor (e.g., α₄β₁ integrin). A chemical ligand specific for α₄β₁ integrin such as, for example, the peptidomimetic Ligand 2A shown above can be linked to an antibody or fragment thereof (e.g., Fc fragment) via non-site specific conjugation to form an antibody conjugate that is suitable for use in imaging a tumor, organ, or tissue in accordance with the present invention. The conjugation can occur, for example, by means of a maleimide functional group present on the targeting agent and a complementary thiol functional group present in the constant region of the antibody or fragment thereof. The conjugation can also occur by means of an oxidized DOPA functional group present on the targeting agent and a complementary amine functional group present in the constant region of the antibody or fragment thereof. Alternatively, the conjugation can occur by means of an aminoxy functional group present on the targeting agent and a complementary ketone functional group present in the constant region of the antibody or fragment thereof. Additional examples of complementary functional groups suitable for the non-site specific covalent attachment of a targeting agent to an antibody or fragment thereof are shown in Table 1. In a preferred embodiment, the complementary functional group is not present at the amino-terminus or carboxyl-terminus of the antibody or fragment thereof, i.e., the complementary functional group is present at an internal site in the antibody molecule.

In some embodiments, the antibody can further comprise a carboxyl-terminal polyarginine linker. Preferably, the polyarginine linker further comprises an imaging agent attached thereto. However, one of skill the art will appreciate that the imaging agent can alternatively be attached to functional groups present at the amino-terminus of the antibody or to functional groups present in its heavy chain constant region.

In certain instances, the imaging agent comprises a radionuclide (e.g., bound to a chelating agent), biotin, a fluorophore, a fluorescent protein, horseradish peroxidase, or alkaline phosphatase. In instances where a radionuclide comprises the imaging agent, detection occurs when radiation from the radionuclide is used to determine where the antibody conjugate is concentrated in the subject. In instances where a fluorophore or fluorescent protein comprises the imaging agent, detection occurs when fluorescence from the fluorophore or fluorescent protein is used to determine where the antibody conjugate is concentrated in the subject.

In a further aspect, the present invention provides a kit for immunotherapy comprising:
(a) a conjugate comprising an antibody having at least a portion of a constant region and a targeting agent comprising a functional group covalently attached to a complementary functional group present in the constant region; and
(b) directions for use of the conjugate in immunotherapy.

In a related aspect, the present invention provides a kit for imaging a tumor, organ, or tissue comprising:
(a) a conjugate comprising an antibody having at least a portion of a constant region and a targeting agent comprising a functional group covalently attached to a complementary functional group present in the constant region; and
(b) directions for use of the conjugate in imaging the tumor, organ, or tissue.

In some embodiments, the kit of the present invention can be used for imaging other types of samples including, but not limited to, a cell, a bioaggregate (i.e., cell aggregate), a biofilm, and the like.

Other objects, features, and advantages of the present invention will be apparent to one of skill in the art from the following detailed description and figures.

### BRIEF DESCRIPTION OF THE DRAWINGS

FIG. 1 shows the structure of a whole IgG antibody molecule and the structures of several antibody fragments of the present invention.

FIG. 2 shows the structures of antibody conjugates that can be generated by non-site specific conjugation of a chemical ligand to various antibody fragments.

FIG. 3 shows the structures of antibody conjugates that can be generated by site-specific conjugation of a chemical ligand to various antibody fragments.

FIG. 4 shows a scheme for the synthesis of a Ligand 2A-DOPA-Biotin targeting agent.

FIG. 5 shows the chemical structures of Ligand 2A-DOPA-Biotin, Ligand 1A-DOPA-Biotin, BIO-1211-DOPA-Biotin, and Ac-LAV-DOPA-Biotin (negative control).

FIG. 6 shows the mass spectrum of IgG1 Fc alone (black line) and IgG1 Fc after conjugation to Ligand 2A-DOPA-Biotin (white line). The Fc antibody conjugate demonstrated a molecular weight shift corresponding to approximately two ligand molecules per Fc fragment.

FIG. 7 shows an electrophoretic analysis of IgG1 Fc alone and IgG1 Fc after conjugation to Ligand 1A-DOPA-Biotin or Ligand 2A-DOPA-Biotin. 10 μg of Fc alone or Fc antibody conjugate were run on a 10% reducing SDS gel. Fc antibody conjugates were probed with 1:200,000 neutravidin-PE (left). The corresponding silver-stained gel is also shown (right).

FIG. 8 shows an immunofluorescent staining analysis of alpha4-transfected K562 cells incubated with 100 nM IgG1 Fc alone or an Fc antibody conjugate described herein. Following incubation and washing, cells were stained with 1:1000 streptavidin-PE or 1:500 anti-Fc FITC.

FIG. 9 shows the ability of the Fc antibody conjugates of the present invention to induce antibody-dependent cell-mediated cytotoxicity. NK cells derived from peripheral blood were stimulated for 5 days with IL-2, harvested, and co-cultured with K562 cells (which do not express alpha4-integrin) or alpha4-transfected K562 cells for 4 h in the presence of 25 nM IgG1 Fc alone or an Fc antibody conjugate described herein. Cytotoxicity was measured by the amount of lactose dehydrogenase (LDH) release.

FIG. 10 shows a scheme for the synthesis of a Ligand 2A-Maleimide targeting agent.

FIG. 11 shows a schematic representation of the site-specific conjugation of Ligand 2A-Maleimide to hIgG1-Cys-Fc.

FIG. 12 shows a MALDI-TOF mass spectrometric analysis of whole hIgG1 and Ligand 2A-hIgG1, which illustrates that approximately 4 Ligand 2A-Maleimide molecules were conjugated to hIgG1.

FIG. 13 shows the chemical structure of a Ligand 2A-Glyoxylyl targeting agent.

FIG. 14 shows cell binding studies illustrating that MOLT-4 cells bound to Ligand 2A-hIgG1-Cys-Fc coated beads, but not to beads coated with a hIgG1-Cys-Fc control.

FIG. 15 shows cell binding studies illustrating that MOLT-4 cells bound to Ligand 2A-hIgG1 (whole IgG1 molecule) coated beads, but not to beads coated with a whole hIgG1 control.

FIG. 16 shows cell killing experiments illustrating that Ligand 2A-hIgG1-Cys-Fc and Ligand 2A-hIgG1 were capable of inducing antibody-dependent cell-mediated cytotoxicity (ADCC).

FIG. 17 shows a scheme for the synthesis of a Ligand 2A-Lys(Aoa) targeting agent.

FIG. 18 shows a scheme for the synthesis of a Ligand 2A-antibody conjugate using a ketone-oxime conjugation method.

### DETAILED DESCRIPTION OF THE INVENTION

### I. Introduction

The present invention provides novel antibody conjugates that advantageously display high binding affinity, specificity, and stability. Importantly, the antibody conjugates (e.g., chemical antibodies) described herein are capable of generating an appropriate immune response by recruiting host immune effector cells and/or activating the host complement system. As a result, the antibody conjugates of the present invention find utility in replacing currently available antibody-based approaches such as targeting, neutralizing, optionizing, clearing, recruiting effector cells, and are particularly useful in diagnostic methods such as in vivo imaging and therapeutic methods such as treatment of a wide variety of diseases and disorders.

As described herein, the antibody conjugates of the present invention can be used as molecular scaffolds for the delivery of multiple therapeutic, imaging, and/or other agents (e.g., cytotoxic or apoptotic agents) to a desired target cell, tumor, tissue, or organ. Other advantages of the antibody conjugates of the present invention include: (1) their ability to target tumors and provide solid tumor penetration, higher total tumor uptake, substantially faster blood clearance, and better tumor-to-blood ratios; (2) their utility in connecting the host immune system (i.e., complement and effector cell systems) to fight against abnormal host cells, cancer, autoimmune cells, infectious agents, pathogens, and the like; (3) their ability to efficiently neutralize cytokines or inhibit cytokine production from inflammatory cells, e.g., when using antibody conjugates that do not contain CH3 constant domains; (4) their increased serum half-life and stability in host blood as compared to monobodies and diabodies, e.g., by being resistant to plasma proteases; (5) their minimal amount of immune reactivity in the host (i.e., lower immunogenicity); and (6) their utility as bispecific and multivalent cancer targeting agents, e.g., having both antigenic and apoptotic targeting properties.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice the present invention. For purposes of the present invention, the following terms are defined.

The term “conjugate” is intended to include a chemical compound that has been formed by the joining or attachment of two or more compounds. In particular, a conjugate of the present invention is an “antibody conjugate” comprising a targeting agent covalently attached to an antibody or fragment thereof (e.g., fragments of the Fc region). Preferably, an antibody conjugate of the present invention is a “chemical antibody” comprising a ligand covalently attached to an antibody or fragment thereof (e.g., Fc fragments).

As used herein, the term “targeting agent” includes compounds that will selectively localize to a particular tumor, tissue, organ, or other region of the body. The localization can be mediated by specific recognition of molecular determinants, the molecular size or weight of the targeting agent or conjugate, ionic interactions, hydrophobic interactions, and the like. Other mechanisms of targeting an agent to a particular tissue or region are known to those of skill in the art. Exemplary targeting agents include, but are not limited to, small organic molecules, peptides, peptidomimetics, peptoids, proteins, polypeptides, glycoproteins, oligosaccharides, nucleic acids, and the like. In certain instances, the targeting agent comprises a compound such as a peptidomimetic that cannot be photolytically, chemically, thermally, and/or enzymatically cleaved, e.g., by a protease. Preferably, the targeting agent comprises a peptide or peptidomimetic ligand specific for an integrin receptor expressed by a particular cell, tumor, tissue, or organ. As a non-limiting example, U.S. Patent Publication No. 20060019900 provides a description of the synthesis and structures of peptidomimetic ligands specific for α₄β₁ integrin (e.g., Ligand 2A, also known as LLP2A) that are suitable for use as targeting agents in the antibody conjugates of the present invention.

The term “ligand” refers to a molecule that is able to bind to and form a complex with a biomolecule to serve a biological purpose. A ligand is generally an effector molecule that binds to a site on a target protein, e.g., by intermolecular forces such as ionic bonds, hydrogen bonds, hydrophobic interactions, dipole-dipole bonds, or Van der Waals forces. Ligands suitable for conjugation to dimeric antibody fragments such as Fc fragments are typically synthetic compounds such as small organic molecules, peptides, peptidomimetics, peptoids, proteins, polypeptides, glycoproteins, oligosaccharides, or nucleic acids that are capable of binding to receptors present on the surface of target cells or to components of the extracellular matrix.

Exemplary ligands include, but are not limited to, molecules that bind to cell-surface receptors such as integrin receptors, lectin receptors, immunoglobulin receptors, receptor tyrosine kinases, growth factor receptors, insulin receptors, fibroblast growth factor receptors, neurotrophin receptors, Eph receptors, G-protein coupled receptors, adrenergic receptors, olfactory receptors, NMDA receptors, Toll-like receptors, T cell receptors, and the like. In some embodiments, the ligand binds to a receptor that is responsible for the abnormal function of a cell, e.g., carcinogenesis. In other embodiments, the ligand binds to a target protein on a pathogen, microbial agent, or infectious agent such as, for example, a virus, bacterium, fungus, parasite, or any other infectious agent. For example, the ligand can bind to a viral coat protein or bacterial cell wall peptidoglycan. In certain embodiments, the ligand binds to a target protein aggregate such as amyloid, prions, and the like. In certain other embodiments, the ligand binds to a toxin or metabolite derived from host metabolism or from a pathogen, microbial agent, or infectious agent. In further embodiments, the ligand binds to a poisonous agent such as snake venom or drugs.

A “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally-occurring amino acids and/or unnatural amino acids, but can also comprise modifications to the peptide backbone. Peptidomimetics, such as the peptidomimetic ligands described herein, can exhibit increased affinity, specificity, and/or stability compared to an existing peptide.

The term “amino acid” includes naturally-occurring a-amino acids and their stereoisomers, as well as unnatural amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid.

Naturally-occurring amino acids are those encoded by the genetic code, as well as those amino acids that are later modified, e.g., γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an a carbon that is bound to a hydrogen, a carboxyl group, an amino group, but have modified R (i.e., side-chain) groups.

Non-limiting examples of unnatural amino acids include 1-aminocyclopentane-1-carboxylic acid (Acp), 1-aminocyclobutane-1-carboxylic acid (Acb), 1-aminocyclopropane-1-carboxylic acid (Acpc), citrulline (Cit), homocitrulline (HoCit), α-aminohexanedioic acid (Aad), 3-(4-pyridyl)alanine (4-Pal), 3-(3-pyridyl)alanine (3-Pal), propargylglycine (Pra), α-aminoisobutyric acid (Aib), α-aminobutyric acid (Abu), norvaline (Nva), α,β-diaminopropionic acid (Dpr), α,γ-diaminobutyric acid (Dbu), α-tert-butylglycine (Bug), 3,5-dinitrotyrosine (Tyr(3,5-di NO₂)), norleucine (Nle), 3-(2-naphthyl)alanine (Nal-2), 3-(1-naphthyl)alanine (Nal-1), cyclohexylalanine (Cha), di-n-propylglycine (Dpg), cyclopropylalanine (Cpa), homoleucine (Hle), homoserine (HoSer), homoarginine (Har), homocysteine (Hcy), methionine sulfoxide (Met(O)), methionine methylsulfonium (Met(S-Me)), α-cyclohexylglycine (Chg), 3-benzo-thienylalanine (Bta), taurine (Tau), hydroxyproline (Hyp), O-benzyl-hydroxyproline (Hyp(Bzl)), homoproline (HoPro), β-homoproline (βHoPro), thiazolidine-4-carboxylic acid (Thz), nipecotic acid (Nip), isonipecotic acid (IsoNip), 3-carboxymethyl-1-phenyl-1,3,8-triazaspiro[4,5]decan-4-one (Cptd), tetrahydro-isoquinoline-3-carboxylic acid (3-Tic), 5H-thiazolo [3,2-a]pyridine-3-carboxylic acid (Btd), 3-aminobenzoic acid (3-Abz), 3-(2-thienyl)alanine (2-Thi), 3-(3-thienyl)alanine (3-Thi), α-aminooctanedioc acid (Asu), diethylglycine (Deg), 4-amino-4-carboxy-1,1-dioxo-tetrahydrothiopyran (Acdt), 1-amino-1-(4-hydroxycyclohexyl)carboxylic acid (Ahch), 1-amino-1-(4-ketocyclohexyl)carboxylic acid (Akch), 4-amino-4-carboxytetrahydropyran (Actp), 3-nitrotyrosine (Tyr(3-NO₂)), 1-amino-1-cyclohexane carboxylic acid (Ach), 1-amino-1-(3-piperidinyl)carboxylic acid (3-Apc), 1-amino-1-(4-piperidinyl)carboxylic acid (4-Apc), 2-amino-3-(4-piperidinyl) propionic acid (4-App), 2-aminoindane-2-carboxylic acid (Aic), 2-amino-2-naphthylacetic acid (Ana), (2S, 5R)-5-phenylpyrrolidine-2-carboxylic acid (Ppca), 4-thiazoylalanine (Tha), 2-aminooctanoic acid (Aoa), 2-aminoheptanoic acid (Aha), omithine (Om), azetidine-2-carboxylic acid (Aca), α-amino-3-chloro-4,5-dihydro-5-isoazoleacetic acid (Acdi), thiazolidine-2-carboxylic acid (Thz(2-COOH)), allylglycine (Agl), 4-cyano-2-aminobutyric acid (Cab), 2-pyridylalanine (2-Pal), 2-quinoylalanine (2-Qal), cyclobutylalanine (Cba), a phenylalanine analog, derivatives of lysine, omithine (Orn) and α,γ-diaminobutyric acid (Dbu), stereoisomers thereof, and combinations thereof (see, e.g., Liu et al., Anal. Biochem., 295:9-16 (2001)). As such, the unnatural α-amino acids are present either as unnatural L-α-amino acids, unnatural D-α-amino acids, or combinations thereof.

“Amino acid mimetics” are chemical compounds that have a structure that is different from the general chemical structure of an amino acid, but that function in a manner similar to a naturally-occurring amino acid. Suitable amino acid mimetics include, without limitation, β-amino acids and γ-amino acids. In β-amino acids, the amino group is bonded to the β-carbon atom of the carboxyl group such that there are two carbon atoms between the amino and carboxyl groups. In γ-amino acids, the amino group is bonded to the γ-carbon atom of the carboxyl group such that there are three carbon atoms between the amino and carboxyl groups. Suitable R groups for β- or γ-amino acids include, but are not limited to, side-chains present in naturally-occurring amino acids and unnatural amino acids.

“N-substituted glycines” are unnatural amino acids based on glycine, where an amino acid side-chain is attached to the glycine nitrogen atom. Suitable amino acid side-chains (e.g., R groups) include, but are not limited to, side chains present in naturally-occurring amino acids and side-chains present in unnatural amino acids such as amino acid analogs. Examples of N-substituted glycines suitable for use in the present invention include, without limitation, N-(2-aminoethyl)glycine, N-(3-aminopropyl)glycine, N-(2-methoxyethyl)glycine, N-benzylglycine, (S)-N-(1-phenylethyl)glycine, N-cyclohexylmethylglycine, N-(2-phenylethyl)glycine, N-(3-phenylpropyl)glycine, N-(6-aminogalactosyl)glycine, N-(2-(3′-indolylethyl)glycine, N-(2-(p-methoxyphenylethyl))glycine, N-(2-(p-chlorophenylethyl)glycine, and N-[2-(p-hydroxyphenylethyl)]glycine. N-substituted glycine oligomers, referred to herein as “peptoids,” have been shown to be protease resistant (see, e.g., Miller et al., Drug Dev. Res., 35:20-32 (1995)).

Amino acids may be referred to herein by either their commonly known three letter symbols or by the one-letter symbols recommended by the IUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins, 1984).

The term “peptide” refers to a compound made up of a single chain of D- or L-amino acids or a mixture of D- and L-amino acids joined by peptide bonds. Generally, peptides are about 2 to about 50 amino acids in length. Preferably, the peptide ligands of the present invention are 2 to 25 amino acids in length, 3 to 20 amino acids in length, or 3 to 7 amino acids in length.

A “functional group” as used herein refers to a specific group of atoms within a molecule that is responsible for the characteristic chemical reactions of that molecule. Examples of functional groups include, but are not limited to, hydroxyl, aldehyde, alkyl, alkenyl, alkynyl, amide, amine, carboxamide, amine (i.e., primary, secondary, tertiary, or quaternary), aminoxy, azide, azo (diimide), benzyl, carbonate ester, carboxyl, cyanate, thiocyanate, ester, ether, glyoxylyl, haloalkyl, haloformyl, imine, imide, ketone, maleimide, isocyanide, isocyanate, carbonyl, nitrate, nitrile, nitrite, nitro, nitroso, peroxide, phenyl, phosphino, phosphate, phosphono, pyridyl, sulfide, sulfonyl, sulfinyl, thioester, thioether, thiol(sulthydryl), and oxidized 3,4-dihydroxy-phenylalanine (DOPA) groups. Additional examples of functional groups suitable for covalently attaching a targeting agent to an antibody or fragment thereof are shown in Table 1, wherein the bond results from a reaction between complementary functional groups present on the targeting agent and the antibody or antibody fragment. One of skill in the art will know of other functional groups suitable for use in the present invention.

The term “linker” or “linking group” refers to a moiety that possesses one or more different functional groups that allows for covalent attachment of a targeting agent to an antibody or fragment thereof. Preferably, the linker possesses two different functional groups, i.e., a heterobifunctional linker. Suitable linkers include, without limitation, those available from Pierce Biotechnology, Inc. (Rockford, Ill.). As a non-limiting example, ε-amino groups present on lysine residues in the constant region of an antibody or fragment thereof can be derivatized with a heterobifunctional linker (e.g., N-succinimidyl levulinic acetate) to provide a complementary functional group (e.g., methyl-ketone group) that can be used to covalently attach a functional group (e.g., aminoxy group) present on a targeting agent. However, one of skill in the art will appreciate that the targeting agent can alternatively be derivatized with a heterobifunctional linker to provide a functional group that can be covalently attached to a complementary functional group present in the constant region of the antibody molecule.

The term “antibody” includes a polypeptide comprising a framework region from an immunoglobulin gene or a fragment thereof. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma (γ), mu (μ), alpha (α), delta (δ), or epsilon (ε), which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a “variable region” of about 100 to 110 or more amino acids primarily responsible for antigen recognition. The remainder of each chain defines a “constant region” that is conserved and exhibits low variability among different antibodies. Each light chain contains a variable region having one variable (VL) domain and a constant region having one constant (CL) domain. Each heavy chain contains a variable region having one variable (VH) domain and a constant region having three or four constant (CH) domains. Different classes of constant regions in the stem of the antibody generate different isotypes with differing properties based on their amino acid sequence.

A “constant domain” refers to an immunoglobulin (Ig) domain of about 70-110 amino acids in the constant region of a light or heavy chain. In general, heavy chains γ, α, and δ have a constant region composed of three tandem constant (CH) domains (i.e., CH1, CH2, CH3), whereas heavy chains μ and ε have a constant region composed of four constant (CH) domains (i.e., CH1, CH2, CH3, CH4). The constant region typically contains a hinge region between the CH1 and CH2 domains, which provides added flexibility and connects the two heavy chains via disulfide bonding. The CH2 domain comprises a complement binding domain that activates the complement system, which is a biochemical cascade that helps clear pathogens from an organism. The CH2 domain also comprises an effector cell binding domain (Fc receptor) that activates effector cells (e.g., natural killer (NK) cells, macrophages, mast cells, neutrophils, etc.) via cell-surface Fc receptors to invoke an appropriate immune response for a particular pathogen.

As used herein, the term “fragment crystallizable region” or “Fc region” refers to the carboxyl-terminal region of an antibody that interacts with cell-surface Fc receptors and certain proteins of the complement system. In the IgG, IgA, and IgD antibody isotypes, the Fc region comprises two identical protein fragments, derived from the second and third constant domains of the antibody's two heavy chains (i.e., CH2 and CH3). IgM and IgE Fc regions contain three heavy chain constant domains (i.e., CH2, CH3, and CH4) in each of the two heavy chains. The Fc region typically includes a hinge region, which covalently links the antibody's two heavy chains by disulfide bonds.

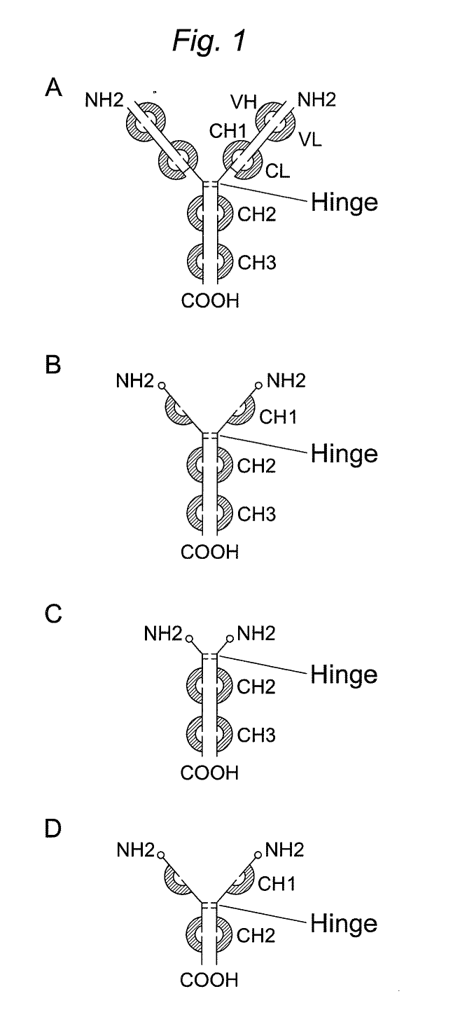

The term “Fc fragment” refers to a dimer of heavy chain fragments in which each heavy chain fragment contains the second and third constant domains of a heavy chain (i.e., CH2 and CH3) and optionally includes a hinge region. In some embodiments, the presence of a hinge region in each of the heavy chain fragments enables dimer formation via disulfide bonding to produce an Fc fragment. Examples of Fc fragments that are commercially available include, but are not limited to, a recombinant human IgG1 Fc fragment from R&D Systems, Inc. (Minneapolis, Minn.); a purified human IgG Fc fragment from Meridian Life Science, Inc. (Saco, Me.), Calbiochem (San Diego, Calif.), and Bethyl Laboratories (Montgomery, Tex.); a purified mouse IgG Fc fragment from Pierce Biotechnology, Inc. (Rockford, Ill.); and a purified human IgD Fc fragment from MorphoSys US Inc. (Raleigh, N.C.). The structure of an exemplary Fc fragment is shown in FIG. 1C.

The term “dimer” refers to a molecule composed of two subunits or monomers linked together, for example, by covalent interactions (e.g., disulfide bonds) or noncovalent interactions (e.g., ionic bonds, hydrophobic interactions, hydrogen bonds, Van der Waals forces, dipole-dipole bonds). Dimers can comprise identical subunits or monomers (i.e., homodimers), or alternatively, different subunits or unrelated monomers (i.e., heterodimers).

The term “therapeutically effective amount” refers to the amount of an antibody conjugate of the present invention that is capable of achieving a therapeutic effect in a subject in need thereof. For example, a therapeutically effective amount of an antibody conjugate of the present invention can be the amount that is capable of preventing or relieving one or more symptoms associated with a disease or disorder. One skilled in the art will appreciate that the antibody conjugates of the present invention can be co-administered with other therapeutic agents such as anticancer, anti-inflammatory, immunosuppressive, antiviral, antibiotic, or antifungal agents.

As used herein, the term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of an antibody conjugate of the present invention for preventing or relieving one or more symptoms associated with cancer or an inflammatory or autoimmune disease. By “co-administer” it is meant that an antibody conjugate of the present invention is administered at the same time, just prior to, or just after the administration of a second drug (e.g., anticancer agent, anti-inflammatory agent, immunosuppressive agent, antiviral agent, antibiotic, antifungal agent, etc.).

The term “radionuclide” is intended to include any nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance. Examples of radionuclides suitable for use in the present invention include, but are not limited to, fluorine 18 (¹⁸F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (64Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90(⁹⁰Y), rhodium 105 (¹⁰⁵Rh), silver 111 (¹¹¹Ag), indium 111 (¹¹¹In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (¹⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹Tl), astatine 211 (²¹¹At), and bismuth 212 (²¹²Bi). As used herein, the “m” in ^{117m}Sn and ^{99m}Tc stands for the meta state. Additionally, naturally-occurring radioactive elements such as uranium, radium, and thorium, which typically represent mixtures of radioisotopes, are suitable examples of radionuclides. 67Cu, ¹³¹I, ¹⁷⁷Lu, and ¹⁸⁶Re are beta- and gamma-emitting radionuclides. ²¹²Bi is an alpha- and beta-emitting radionuclide. ²¹¹At is an alpha-emitting radionuclide. ³²P, ⁴⁷Sc, ⁸⁹Sr, ⁹⁰Y, ¹⁰⁵Rh, ¹¹¹Ag, ^{117m}Sn, ¹⁴⁹Pm, ¹⁵³Sm, ¹⁶⁶Ho, and ¹⁸⁸Re are examples of beta-emitting radionuclides. ⁶⁷Ga, ¹¹¹In, ^{99m}Tc, and ²⁰¹Tl are examples of gamma-emitting radionuclides. ⁵⁵Co, ⁶⁰Cu, ⁶¹Cu, ⁶²Cu, ⁶⁶Ga, ⁶⁸Ga, ⁸²Rb, and ⁸⁶Y are examples of positron-emitting radionuclides. ⁶⁴Cu is a beta- and positron-emitting radionuclide.

The term “chelating agent” refers to a compound which binds to a metal ion, such as a radionuclide, with considerable affinity and stability. In addition, chelating agents can be bifunctional, having a metal ion chelating group at one end and a reactive functional group capable of binding to peptides, peptidomimetics, polypeptides, or proteins at the other end. Examples of bifunctional chelating agents include, but are not limited to, 1,4,7,10-tetraazacyclododecane-N,N′,N″,N′″-tetraacetic acid (DOTA), a bromoacetamidobenzyl derivative of DOTA (BAD), 1,4,8,11-tetraazacyclotetradecane-N,N′,N″,N′″-tetraacetic acid (TETA), diethylenetriaminepentaacetic acid (DTPA), the dicyclic dianhydride of diethylenetriaminepentaacetic acid (ca-DTPA), 2-(p-isothiocyanatobenzyl)-diethylenetriaminepentaacetic acid (SCNBzDTPA), and 2-(p-isothiocyanatobenzyl)-5(6)-methyl-diethylenetriaminepentaacetic acid (MxDTPA) (see, e.g., Ruegg et al., Cancer Research, 50:4221-4226 (1990); DeNardo et al., Clinical Cancer Research, 4:2483-2490 (1998)). Other chelating agents include EDTA, NTA, HDTA and their phosphonate analogs such as EDTP, HDTP, NTP (see, e.g., Pitt et al., “The Design of Chelating Agents for the Treatment of Iron Overload,” In INORGANIC CHEMISTRY IN BIOLOGY AND MEDICINE, Martell Ed., American Chemical Society, Washington, D.C., pp. 279-312 (1980); Lindoy, THE CHEMISTRY OF MACROCYCLIC LIGAND COMPLEXES, Cambridge University Press, Cambridge (1989); Dugas, BIOORGANIC CHEMISTRY, Springer-Verlag, New York (1989); and references contained therein).

The term “subject” or “patient” typically refers to humans, but can also include other animals such as, e.g., other primates, rodents, canines, felines, equines, ovines, porcines, and the like.

### III. Description of the Embodiments

The present invention provides, inter alia, antibody conjugates comprising a targeting agent covalently attached to an antibody or fragment thereof. The antibody conjugates of the present invention are particularly useful for imaging a tumor, organ, or tissue and for treating diseases and disorders such as cancer, inflammatory diseases, autoimmune diseases, infectious diseases, and neurological disorders. Kits containing the antibody conjugates described herein find utility in a wide range of applications including, for example, in vivo imaging and immunotherapy.

FIG. 1A shows the structure of a whole IgG antibody molecule. As shown therein, the intact IgG is a tetramer composed of two identical pairs of polypeptide chains, each pair having one light chain and one heavy chain. Each light chain contains a variable region having one antigen-binding variable (VL) domain and a constant region having one constant (CL) domain. Each heavy chain contains a variable region having one antigen-binding variable (VH) domain and a constant region having three constant (CH) domains (i.e., CH1, CH2, and CH3).

FIGS. 1B-1D provide an illustration of several embodiments of the antibody fragments of the present invention. The antibody fragments generally comprise at least a portion of a constant region, e.g., CH1, CH2, CH3, and/or CH4 constant domains, and can optionally include a hinge region. However, one of skill in the art will recognize other variations, modifications, and alternatives. In particular, FIG. 1B illustrates an IgG antibody fragment comprising a dimer of identical heavy chain fragments, each containing CH1, CH2, and CH3 constant domains and a hinge region. This dimeric antibody molecule, which does not comprise any light chains or variable (VH) domains, has a molecule weight of about 90-100 kDa. FIG. 1C illustrates an IgG antibody fragment comprising a dimer of identical heavy chain fragments, each containing CH2 and CH3 constant domains and a hinge region. This dimeric antibody molecule, which does not comprise any light chains, variable (VH) domains, or CH1 constant domains, has a molecule weight of about 50-60 kDa. FIG. 1D illustrates an IgG antibody fragment comprising a dimer of identical heavy chain fragments, each containing CH1 and CH2 constant domains and a hinge region. This dimeric antibody molecule, which does not comprise any light chains, variable (VH) domains, or CH3 constant domains, has a molecule weight of about 50-60 kDa. Although examples of homodimeric antibody fragments have been described, one of skill in the art will appreciate that heterodimers comprising different heavy chain fragments of the same or different antibody class or isotype are encompassed within the scope of the present invention.

In certain aspects, the antibody component further comprises a targeting moiety covalently attached thereto, thereby forming an antibody conjugate of the present invention. Non-limiting examples of targeting agents are described herein and include small organic molecules, peptides, peptidomimetics, peptoids, proteins, polypeptides, glycoproteins, oligosaccharides, nucleic acids, and the like. Preferably, the targeting agent comprises a ligand specific for an integrin receptor (e.g., α₄β₁ integrin peptide or peptidomimetic ligand) expressed by a particular cell, tumor, tissue, or organ. One of skill in the art will appreciate that ligands specific for other integrin receptors (e.g., α₅β₁, αᵥβ₃, αᵥβ₅, etc.) are also suitable for conjugation to the antibodies or antibody fragments described herein.

In some embodiments, the targeting agent can be synthesized using peptide synthesis techniques known in the art by incorporating naturally-occurring and/or unnatural amino acids into the sequence of the resulting peptide or peptidomimetic and chemically fused (i.e., conjugated) to the antibody fragment. In other embodiments, the targeting agent can be synthesized as a small molecule using art-recognized techniques and chemically fused (i.e., conjugated) to the antibody fragment. One of skill in the art will understand that different types or classes of targeting agents, directed to the same target or to several different targets, can be covalently attached to the antibody or antibody fragment, such that antibody conjugates with multiple specificities (e.g., bispecific, trispecific, and the like) can be produced.

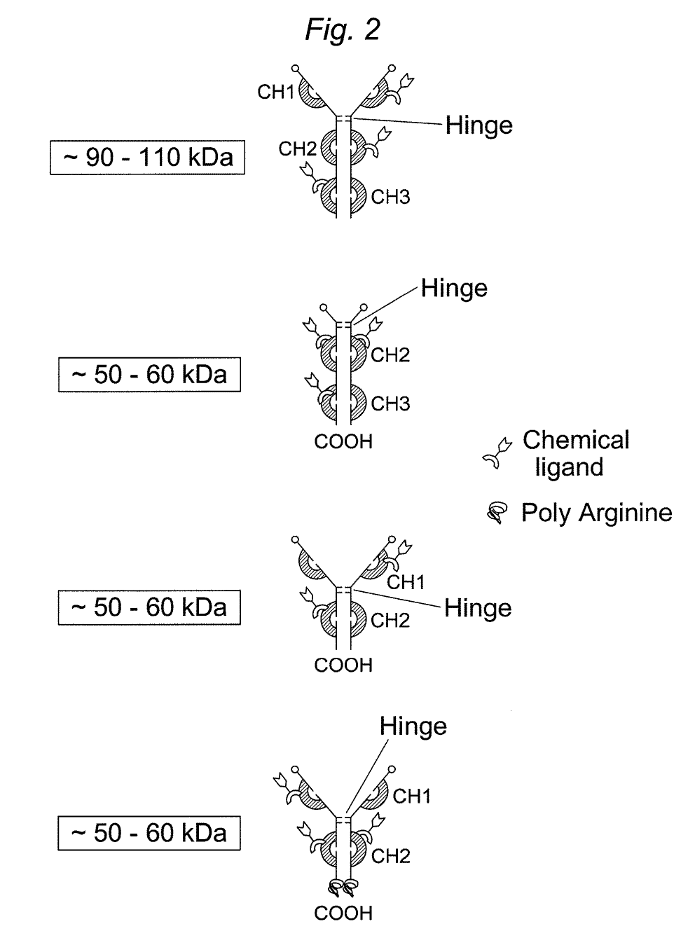

In certain embodiments, the targeting agent is attached to the antibody or fragment thereof via non-site specific conjugation. As a non-limiting example, antibody conjugates can be generated by covalent attachment of a targeting agent comprising a functional group to a complementary functional group (e.g., thiol group, primary amine group, or any other functional group) present in the constant region (e.g., CH1, CH2, CH3, and/or CH4 domains and/or the hinge region) of the antibody or fragment thereof. Alternatively, antibody conjugates can be generated by covalent attachment of a targeting agent comprising a functional group to a complementary functional group (e.g., methyl-ketone group or any other functional group) present in a linker attached to the side-chain of an amino acid present in the constant region of the antibody molecule. Preferably, the conjugation occurs at positions on the targeting agent and antibody component that do not interfere with their functions. FIG. 2 provides an illustration of several embodiments of antibody conjugates that can be generated by non-site specific conjugation of ligands (e.g., chemical ligands such as peptides, peptidomimetics, or small organic molecules) comprising functional groups to complementary functional groups present in the CH1, CH2, and/or CH3 constant domains of antibody fragments containing at least a portion of a constant region.

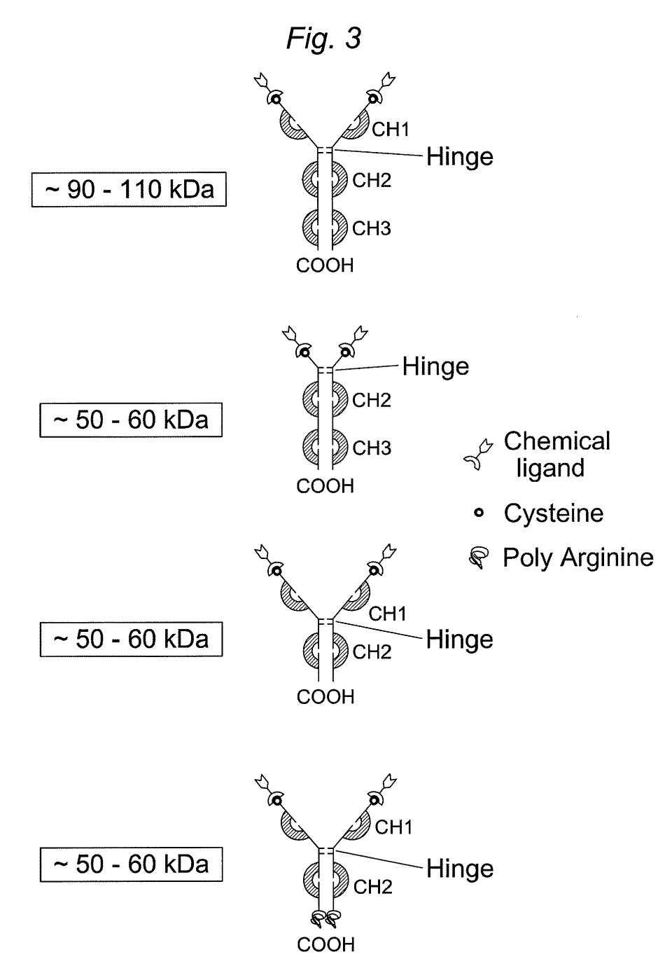

In certain other embodiments, the targeting agent can be attached to the antibody or fragment thereof via site-specific conjugation. For example, antibody conjugates can be generated by covalent attachment of a targeting agent comprising a functional group to an amino acid (e.g., cysteine) present at one or both of the two amino-terminal ends of the antibody or fragment thereof. In one embodiment, a stretch of one, two, three, four, five, six, seven, or more cysteines can be introduced at each amino-terminus of the antibody component by site-directed mutagenesis or by covalently attaching a polycysteine linker thereto using methods known to one of skill in the art. Preferably, the conjugation occurs at positions on the targeting agent and antibody component that do not interfere with their functions. FIG. 3 provides an illustration of several embodiments of antibody conjugates that can be generated by site-specific conjugation of ligands (e.g., chemical ligands such as peptides, peptidomimetics, or small organic molecules) comprising functional groups to cysteine residues present at each of the two amino-termini of the antibody or fragment thereof.

In certain other aspects, the antibody conjugate further comprises an imaging agent bound via covalent or noncovalent attachment to the targeting agent and/or antibody portion of the antibody conjugate. In some instances, the imaging agent can be attached to the antibody or fragment thereof via site-specific conjugation. As a non-limiting example, an imaging agent can be covalently attached to an amino acid (e.g., arginine) in a linking group (e.g., polyarginine moiety) present at one or both of the carboxyl-terminal ends of the antibody or fragment thereof. In one embodiment, a stretch of one, two, three, four, five, six, seven, or more arginines can be introduced at each carboxyl-terminus of the antibody component by site-directed mutagenesis or by covalently attaching a polyarginine linker thereto using methods known to one of skill in the art. In other instances, the imaging agent can be attached to the targeting agent and/or antibody portion of the antibody conjugate via non-site specific conjugation. For example, an imaging agent can be covalently attached to thiol groups, primary amine groups, or other reactive functional groups present in the constant region of the antibody or fragment thereof. In a preferred embodiment, the conjugation occurs at positions on the imaging agent and antibody component that do not interfere with their functions.

In addition to imaging agents, the antibody conjugates of the present invention can be linked to other agents such as radionuclides, small molecule toxins, chemotherapeutic agents, anticancer agents, nanoparticles, quantum dots, or nanodroplets of an anticancer agent. Such agents can be conjugated, for example, to one or both carboxyl-terminal ends of the antibody portion of the antibody conjugate, to one or both amino-terminal ends of the antibody portion of the antibody conjugate, or to the constant region of the antibody portion of the antibody conjugate. One of skill in the art will also appreciate that targeting agents can be conjugated to one or both carboxyl-terminal ends of the antibody portion of the antibody conjugate, e.g., via arginine residues. One of skill in the art will further understand that the imaging agent can be conjugated to one or both amino-terminal ends of the antibody portion of the antibody conjugate, e.g., via cysteine residues.

### IV. Production of Antibodies and Antibody Fragments

The generation and selection of antibodies and fragments thereof not already commercially available can be accomplished by any method known in the art. Preferably, whole antibody molecules or dimers of antibody heavy chain fragments having at least two constant (CH) domains on each heavy chain fragment (e.g., Fc fragments) are produced and used in preparing the antibody conjugates of the present invention.

Any technique known in the art can be used to prepare whole antibodies, e.g., recombinant, monoclonal, or polyclonal antibodies. See, e.g., Kohler et al., Nature, 256:495-497 (1975); Kozbor et al., Immunology Today, 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice, 2d ed. (1986). In certain instances, the genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells.

Techniques for the production of single chain antibodies or recombinant antibodies can also be adapted to produce antibodies (see, e.g., U.S. Pat. Nos. 4,816,567 and 4,946,778). Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016; Marks et al., Biotechnology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); and Lonberg et al., Intern. Rev. Immunol., 13:65-93 (1995)).

In some embodiments, antibody fragments can be produced by proteolysis of whole antibodies using a peptidase such as papain, plasmin, pepsin, or trypsin. For example, incubation of whole antibodies from the IgA, IgD, IgE, IgG, or IgM class with papain produces two Fab fragments and one Fc fragment. The Fc fragment can be separated from the Fab fragments using an affinity ligand such as Protein A or Protein G, or by ion exchange chromatography (see, e.g., Rousseaux et al., J. Immunol. Meth., 64:141-146 (1983)).

In other embodiments, antibody fragments can be expressed using recombinant DNA methodology in eukaryotic host cells such as baculovirus-infected insect cells (see, e.g., Björklund et al., Mol. Immunol., 37:169-177 (2000)) or a mammalian cell line (see, e.g., Basu et al., J. Biol. Chem., 268:13118-13127 (1993)). Specific examples of eukaryotic host cells include, but are not limited to, Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, and the C127, 3T3, 293, 293T, HeLa, MDCK, and BHK cell lines.

In further embodiments, antibody heavy chain fragments (e.g., Fc-SH fragments) can be expressed and purified from prokaryotic host cells and coupled (e.g., via disulfide bonding) to form dimers (e.g., Fc fragments). Non-limiting examples of prokaryotic host cells include Escherichia coli, Salmonella typhimurium, Bacillus subtilis, and various species within the genera Pseudomonas, Streptomyces, and Staphylococcus. Monomeric antibody heavy chain fragments can also be chemically synthesized de novo and coupled to form dimeric molecules.

One skilled in the art will recognize that alternative procedures are available for the production of antibodies and fragments thereof, as described in, e.g., Antibodies, A Laboratory Manual, Harlow and Lane, Eds., Cold Spring Harbor Laboratory, Cold Spring Harbor, N.Y. (1988) and Antibody Engineering: A Practical Approach, Borrebaeck, Ed., Oxford University Press, Oxford (1995).

When using recombinant techniques, antibodies or fragments thereof (e.g., Fc fragments) can be produced inside an isolated host cell, in the periplasmic space of a host cell, or directly secreted from a host cell into the medium. If the antibody or antibody fragment is produced intracellularly, the particulate debris can first be removed, e.g., by centrifugation or ultrafiltration. In some instances, antibodies or antibody fragments secreted into the periplasmic space of E. coli can be isolated. Briefly, cell paste is thawed in the presence of sodium acetate (pH 3.5), EDTA, and PMSF for about 30 minutes. Cell debris can be removed by centrifugation. Where the antibodies or antibody fragments are secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibodies or antibody fragments prepared from cells can be purified using, for example, hydroxylapatite chromatography, gel electrophoresis, dialysis, and affinity chromatography. In some embodiments, an affinity ligand such as Protein A can be used to purify antibody or antibody fragments including IgG1, IgG2, IgG4, and Fc fragments thereof (see, e.g., Lindmark et al., J. Immunol. Meth., 62:1-13 (1983)). In other embodiments, an affinity ligand such as Protein G can be used to purify antibody or antibody fragments including IgG3 and Fc fragments thereof (see, e.g., Guss et al., EMBO J., 5:1567-1575 (1986)). The matrix to which the affinity ligand is attached is most often agarose, but other matrices are available. For example, mechanically stable matrices such as controlled pore glass or poly(styrenedivinyl)benzene allow for faster flow rates and shorter processing times than can be achieved with agarose. Where the antibody or antibody fragment comprises a CH3 domain, the Bakerbond ABX™ resin (J. T. Baker; Phillipsburg, N.J.) can be useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody or fragment thereof to be recovered.

Following any preliminary purification step(s), the mixture comprising the antibody or antibody fragment of interest and contaminants may be subjected to low pH hydrophobic interaction chromatography using an elution buffer at a pH between about 2.5-4.5, preferably performed at low salt concentrations (e.g., from about 0-0.25 M salt).

### V. Screening Methods

In some aspects, targeting agents such as ligands which bind to biomolecules on target cells, tumors, organs, or tissues can be identified using any screening method known in the art. Targeting agents of interest can either be synthetic or naturally-occurring.

Screening assays can be carried out in vitro or in vivo. Typically, initial screening assays are carried out in vitro, and can be confirmed in vivo using cell-based assays or animal models. The screening methods are designed to screen large chemical or polymer libraries comprising, e.g., small organic molecules, peptides, peptidomimetics, peptoids, proteins, polypeptides, glycoproteins, oligosaccharides, or polynucleotides such as inhibitory RNA (e.g., siRNA, antisense RNA), by automating the assay steps and providing compounds from any convenient source to the assays, which are typically run in parallel (e.g., in microtiter formats on microtiter plates in robotic assays).

The present invention also provides in vitro assays in a high-throughput format. In high-throughput assays, it is possible to screen up to several thousand different compounds in a single day. In particular, each well of a microtiter plate can be used to run a separate assay against a selected potential compound, or, if concentration or incubation time effects are to be observed, every 5-10 wells can test a single compound. Thus, a single standard microtiter plate can assay about 100 (96) compound. If 1536-well plates are used, then a single plate can easily assay from about 100 to about 1500 different compounds. It is possible to assay many different plates per day; assay screens for up to about 6000-20,000, and even up to about 100,000-1,000,000 different compounds are possible using the integrated systems of the present invention. The steps of labeling, addition of reagents, fluid changes, and/or detection are compatible with full automation, for instance, using programmable robotic systems or “integrated systems” commercially available, for example, through BioTX Automation (Conroe, Tex.), Qiagen, Inc. (Valencia, Calif.), Beckman Coulter (Fullerton, Calif.), and Caliper Life Sciences (Hopkinton, Mass.).

Essentially, any chemical compound can be tested as a potential targeting agent for use in the compositions and methods of the present invention. Most preferred are generally compounds that can be dissolved in aqueous or organic (especially DMSO-based) solutions. It will be appreciated that there are many suppliers of chemical compounds, including Sigma-Aldrich (St. Louis, Mo.) and Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

In some embodiments, targeting agents are identified by screening a combinatorial library containing a large number of potential therapeutic compounds. Such “combinatorial libraries” can be screened in one or more assays, as described herein, to identify those library members (particular chemical species or subclasses) that display a desired characteristic activity. The compounds thus identified can serve as conventional “lead compounds” or can themselves be used as potential or actual targeting agents.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

The preparation and screening of combinatorial chemical libraries is well known to those of skill in the art (see, e.g., Beeler et al., Curr Opin Chem Biol., 9:277 (2005); and Shang et al., Curr Opin Chem Biol., 9:248 (2005)). Libraries of use in the present invention can be composed of amino acid compounds, nucleic acid compounds, carbohydrates, or small organic compounds. Carbohydrate libraries have been described in, for example, Liang et al., Science, 274:1520-1522 (1996); and U.S. Pat. No. 5,593,853.

Representative amino acid compound libraries include, but are not limited to, peptide libraries (see, e.g., U.S. Pat. Nos. 5,010,175; 6,828,422; and 6,844,161; Furka, Int. J. Pept. Prot. Res., 37:487-493 (1991); Houghton et al., Nature, 354:84-88 (1991); and Eichler, Comb Chem High Throughput Screen., 8:135 (2005)), peptoids (PCT Publication No. WO 91/19735), encoded peptides (PCT Publication No. WO 93/20242), random bio-oligomers (PCT Publication No. WO 92/00091), vinylogous polypeptides (Hagihara et al., J. Amer. Chem. Soc., 114:6568 (1992)), nonpeptidal peptidomimetics with β-D-glucose scaffolding (Hirschmann et al., J. Amer. Chem. Soc., 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT Application No. PCT/US96/10287; and Vaughn et al., Nature Biotechnology, 14:309-314 (1996)), and peptidyl phosphonates (Campbell et al., J. Org. Chem., 59:658 (1994)).

Representative nucleic acid compound libraries include, but are not limited to, genomic DNA, cDNA, mRNA, inhibitory RNA (e.g., RNAi, siRNA), and antisense RNA libraries. See, e.g., Ausubel, Current Protocols in Molecular Biology, eds. 1987-2005, Wiley Interscience; and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference, or siRNA libraries, are described in, for example, Downward, Cell, 121:813 (2005) and Akashi et al., Nat. Rev. Mol. Cell Biol., 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines, and dipeptides (Hobbs et al., Proc. Nat. Acad. Sci. USA, 90:6909-6913 (1993)); analogous organic syntheses of small compound libraries (Chen et al., J. Amer. Chem. Soc., 116:2661 (1994)); oligocarbamates (Cho et al., Science, 261:1303 (1993)); benzodiazepines (e.g., U.S. Pat. No. 5,288,514; and Baum, C&EN, January 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

Devices for the preparation of combinatorial libraries are commercially available. See, e.g., 357 MPS and 390 MPS from Advanced Chem. Tech (Louisville, Ky.), Symphony from Rainin Instruments (Woburn, Mass.), 433A from Applied Biosystems (Foster City, Calif.), and 9050 Plus from Millipore (Bedford, Mass.).

In a preferred embodiment, targeting agents such as ligands useful for covalent attachment to whole antibodies or dimeric antibody heavy chain fragments are identified using the “one-bead one-compound” (OBOC) combinatorial library method. The OBOC combinatorial library method was first described by Lam et al., Nature, 354:82-84 (1991). In essence, when a “split-mix” synthesis method (Lam et al., supra; Houghten et al., Nature, 354:84-86 (1991); Furka et al., Int. J. Peptide Protein Res., 37:487-493 (1991)) is used to generate a combinatorial library, each bead expresses only one chemical entity (Lam et al., supra; Lam et al., Chem. Rev., 97:411-448 (1997)). Random libraries of millions of beads can then be screened in parallel for a specific acceptor molecule (e.g., receptor, antibody, enzyme, virus, whole cell, etc.). Using an enzyme-linked colorimetric assay similar to that used in Western blotting, the OBOC combinatorial library method was successful in identifying ligands for an anti-β-endorphin antibody (Lam et al., Bioorg. Med. Chem. Lett., 3:419-424 (1993)), streptavidin (Lam et al., Pept.: Chem., Struct., Biol., Proc. Am. Pept. Symp. 13th, pp. 1005-1006 (1994)), avidin (Lam and Lebl, ImmunoMethods, 1:11-15 (1992)), an anti-insulin monoclonal antibody recognizing a discontinuous epitope (Lam et al., In “Peptides: Chem., Sturct., and Biol.” Ed. Hodges, pp. 1003-1004 (1994)), MHC-Class I molecules (Smith et al., Mol. Immunol., 31:1431-1437 (1994)), indigo carmine (a small organic dye) (Lam et al., Drug Dev. Res., 33:157-160 (1994)), and a surface idiotype of B-cell lymphoma cell lines (Lam et al., Biomed. Pept, Prot., and Nuc. Acids, 1:205-210 (1995)). The positive beads were then physically isolated for structural determination by microsequencing using automatic Edman degradation (Lam et al., Nature, 354:82-84 (1991)).

The OBOC combinatorial library method can also be used for screening radiolabeled peptides. For example, substrate motifs for protein kinases were identified using peptides radiolabeled with [γ-³²P]-ATP. (Lam and Wu, Methods, 6:401-403 (1994); Wu et al., Biochem., 33:14825-14833 (1994); Lam et al., Intl. J. Prot. Pept. Res., 45:587-592 (1995); Lou et al., Bioorg. Med. Chem., 4:677-682 (1996)). Using these peptide substrates as templates, potent pseudo-substrate-based peptide inhibitors for p60^{c-src} protein tyrosine kinase were also developed (Alfaro-Lopez et al., J. Med. Chem., 41:2252-2260 (1998)). Since the OBOC combinatorial library method uses a parallel approach, each compound is spatially separated on individual beads, and multiple different peptide motifs can be identified (Wu et al., J. Comb. Chem. High-throughput screening (2002)). Recently, OBOC combinatorial peptidomimetic libraries were used to identify peptidomimetic substrates for the development of c-src inhibitors (Kamath et al., In “Peptides: the wave of the future.” Proc. of Pept. Symp., Jun. 9-14, 2001).

For example, U.S. Patent Publication No.20060019900 describes the synthesis and structures of peptidomimetic ligands specific for α₄β₁ integrin. In particular, using 4-((N′-2-methylphenyl)ureido)-phenylacetyl-LDVP (“BIO-1211⇄) as a template, various OBOC combinatorial peptidomimetic libraries containing both naturally-occurring amino acids, unnatural amino acids, and D-amino acids were designed to elucidate α₄β₁ integrin ligands with increased affinity, specificity, and stability. In order to remove ligands with low to moderate binding affinity, the screening method was modified by incorporating BIO-1211 as a competitive ligand in solution. As a result, only those ligands with high affinity were completely covered by a monolayer of live lymphoid cancer cells. Cancer cell-binding affinity was performed on Jurkat T leukemia cells, Molt-4 leukemia cells, and/or fresh cancer cells obtained from acute lymphocytic leukemia patients. By using this method, α₄β₁ integrin ligands with affinity significantly higher than that of BIO-1211 were identified. Furthermore, the ligands identified by this method contained at least one unnatural α-amino acid, D-amino acid, or a combination thereof, a property that confers greater stability to the ligands upon administration. Therefore, these ligands have significantly better pharmacokinetic properties as well as cancer targeting properties compared to BIO-1211.

Ligands specific for additional members of the integrin family or other cell-surface receptors can also be identified using the OBOC combinatorial library method. Examples of additional integrin family members for which ligands can be identified include, without limitation, α₁β₁, α₂β₁, α₃β₁, α₄β₁, α₄β₇, α₅β₁, α₆β₁, α₆β₄, α₇β₁, α₈β₁, α₉β₁, α_{D}β₂, α_{L}β₂, α_{M}β₂, αᵥβ₁, αᵥβ₃, αᵥβ₅, αᵥβ₆, αᵥβ₈, αₓβ₂, α_{IIb}β₃, and α_{IELb}β₇. Non-limiting examples of other cell-surface receptors for which ligands can be identified include CD19, CD20, CD22, CD37, CD40, L6, CD2, CD28, CD30, CD40, CD50 (ICAM3), CD54 (ICAM1), CD80, CD86, B7-H1, CD134 (OX40), CD137 (41BB), CD152 (CTLA-4), CD153 (CD30 ligand), CD154 (CD40 ligand), ICOS, CD19, CD3, CD4, CD25, CD8, CD11b, CD14, CD25, CD56, CD69, EGFR/HER1/ErbB1, HER2/Neu/ErbB2, HER3/ErbB3, HER4/ErbB4, VEGFR-1/FLT-1, VEGFR-2/FLK-1/KDR, VEGFR-3/FLT-4, FLT-3/FLK-2, PDGFRA, PDGFRB, c-KIT/SCFR, INSR (insulin receptor), IGF-IR, IGF-IIR, IRR (insulin receptor-related receptor), CSF-1R, FGFR-1, FGFR-2, FGFR-3, FGFR-4, HGFR-1, HGFR-2, CCK4, TRK-A, TRK-B, TRK-C, MET, RON, EPHA-1, EPHA-2, EPHA-3, EPHA-4, EPHA-5, EPHA-6, EPHA-7, EPHA-8, EPHB-1, EPHB-2, EPHB-3, EPHB-4, EPHB-5, EPHB-6, AXL, MER, TYRO3, TIE-1, TIE-2, TEK, RYK, DDR-1, DDR-2, RET, c-ROS, LTK (leukocyte tyrosine kinase), ALK (anaplastic lymphoma kinase), ROR-1, ROR-2, MUSK, CD28, and RTK 106, as well as neurotrophin receptors, G-protein coupled receptors, adrenergic receptors, olfactory receptors, NMDA receptors, Toll-like receptors, T cell receptors, and the like.

Ligands specific for other targets can also be identified using the OBOC combinatorial library method. Examples include, but are not limited to, ligands that bind to target proteins on pathogens, microbial agents, or infectious agents (e.g., viruses, bacteria, fungi, parasites, etc.); ligands that bind to target protein aggregates such as amyloid or prion aggregates or any other proteinaceous aggregate associated with a neurological disorder; ligands that bind to target toxins or metabolites derived from a subject's metabolism or from the metabolism of a pathogen, microbial agent, or infectious agent; and ligands that bind to target poisonous agents such as snake venom or drugs.

### VI. Conjugation of Targeting Agents to Antibodies and Antibody Fragments

The attachment of targeting agents to whole antibodies or dimeric antibody heavy chain fragments can be accomplished in several ways. One way is to directly attach a functional group present in a non-interfering position on a targeting agent to a complementary functional group present on an antibody or fragment thereof. Another way is to attach a functional group present in a non-interfering position on a targeting agent to a complementary functional group present on an antibody or fragment thereof using, e.g., commercially available bifunctional linking groups (generally heterobifunctional linkers). Suitable bifunctional linkers include, without limitation, those available from Pierce Biotechnology, Inc. (Rockford, Ill.). One of skill in the art will appreciate that a targeting agent can also be conjugated to an antibody or fragment thereof via noncovalent interactions (e.g., ionic bonds, hydrophobic interactions, hydrogen bonds, Van der Waals forces, dipole-dipole bonds). In this manner, the targeting agent can be used to carry the antibody molecule to a target site (e.g., tumor or organ or tissue having cancerous cells) for diagnostic, therapeutic, or imaging applications.

In one embodiment, the targeting agent is a small organic molecule, peptide, peptidomimetic, peptoid, protein, polypeptide, glycoprotein, oligosaccharide, or nucleic acid. Preferably, the targeting agent is a peptide or peptidomimetic ligand specific for an integrin receptor. One of ordinary skill in the art will recognize other variations, modifications, and alternatives to the targeting agent.

In certain instances, the targeting agent comprises a functional group that is covalently attached to a complementary functional group present in a constant region of the antibody component of the antibody conjugate. Preferably, the functional group on the targeting agent comprises a maleimide group and the complementary functional group comprises one or more thiol groups present in the heavy chain constant domains of the antibody or antibody fragment. Alternatively, the functional group on the targeting agent comprises an oxidized 3,4-dihydroxy-phenylalanine (DOPA) group and the complementary functional group comprises one or more primary amine groups present in the heavy chain constant domains of the antibody or antibody fragment.

In certain other instances, the targeting agent comprises a functional group that is covalently attached to a complementary functional group present at the amino-terminus of the antibody or fragment thereof. Preferably, the functional group on the targeting agent comprises a maleimide or a glyoxylyl group and the complementary functional group comprises the thiol group(s) of one or more cysteine residues present at the amino-terminal end of the antibody or fragment thereof.

Selected examples of reactive functional groups useful for covalently attaching a targeting agent to an antibody or fragment thereof are shown in Table 1, wherein the bond results from a reaction between complementary functional groups present on the targeting agent and the antibody or antibody fragment. Those of skill in the art will know of other linkages suitable for use in the present invention.

TABLE 1

| Reactive Functional Group | Complementary Functional Group | |
|---|---|---|
| (either on the targeting agent or on | (either on the antibody or fragment | |
| the antibody or fragment thereof) | thereof or on the targeting agent) | Resulting Bond |
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| anhydrides | alcohols/phenols | esters |
| anhydrides | amines/anilines | carboxamides/imides |
| aryl halides | thiols | thiophenols |
| aryl halides | amines | aryl amines |
| aziridines | thiols | thioethers |
| boronates | glycols | boronate esters |
| activated carboxylic acids | amines/anilines | carboxamides |
| activated carboxylic acids | alcohols | esters |
| activated carboxylic acids | hydrazines | hydrazides |
| carbodiimides | carboxylic acids | N-acylureas or anhydrides |
| diazoalkanes | carboxylic acids | esters |
| epoxides | thiols (amines) | thioethers (alkyl amines) |
| epoxides | carboxylic acids | esters |
| glyoxylyls | thiols | thiazolidines |
| haloacetamides | thiols | thioethers |
| haloplatinates | amines | platinum complex |
| haloplatinates | heterocycles | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonyl halides | amines/anilines | sulfonamides |

*Activated esters, as understood in the art, generally have the formula —COM, where M is a good leaving group (e.g. succinimidyloxy (—OC₄H₄O₂) sulfosuccinimidyloxy (—OC₄H₃O₂SO₃H), -1-oxybenzotriazolyl (—OC₆H₄N₃); 4-sulfo-2,3,5,6-tetrafluorophenyl; or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates.

In other embodiments, a targeting agent can be attached to an antibody or fragment thereof via a linking group. In certain instances, the targeting agent can be derivatized with a heterobifunctional linker to provide a functional group that can be covalently attached to a complementary functional group present in the constant region of the antibody or fragment thereof. Alternatively, the antibody molecule can be derivatized with a heterobifunctional linker to provide a complementary functional group that can be covalently attached to a functional group present on the targeting agent.

Typically, the functional group on the targeting agent (whether present on a linker or other organic molecule attached thereto) and the complementary functional group on the antibody molecule (whether present on a linker attached thereto or on an amino acid residue of the antibody molecule, e.g., side-chain functional groups such as the ε-amino group of lysine, the thiol group of cysteine, or the carboxyl group of aspartic acid or glutamic acid) comprise an electrophile-nucleophile pair. In an electrophile-nucleophile pair, the electrophile can be, for example, a ketone, an aldehyde, an anhydride, an ester, or an a-halo carbonyl. In these pairings, the nucleophile can be, for example, an amine, a thiol, an alcohol, a hydrazide, an aminoxy group, a thiosemicarbazide, a β-amino thiol, a carboxylate, a thiocarboxylate, or phosphorous-based or carbon-based nucleophiles. In certain instances, the targeting agent can comprise the nucleophile, and the antibody species can comprise the electrophile. In certain other instances, the targeting agent can comprise the electrophile, and the antibody species can comprise the nucleophile. Additional examples of electrophiles and nucleophiles useful in the present invention are described in Lemieux et al., Trends in Biotechnology, 16:506 (1998); and Shin et al., Bull. Korean Chem. Soc., 21:845 (2000).

In a preferred embodiment, a targeting agent comprising an aminoxy functional group is covalently attached to a complementary ketone functional group present on a heterobifunctional linker that is attached to an ε-amino group present on a lysine residue in the constant region of an antibody or fragment thereof. This conjugation method, known as ketone-oxime conjugation, is described, e.g., in U.S. Patent Publication Nos. 20040235027 and 20050255042.

In further embodiments, a targeting agent can be attached to an antibody or fragment thereof via a cleavable linking group. Generally, the cleavable linker connects the targeting agent to the antibody component. The cleavable linker comprises at least one cleavable moiety and one or more optional linker moieties. The cleavable moiety comprises at least one functional group that can be cleaved to allow detachment of the targeting agent from the antibody component. The optional linker moieties typically comprise one or more linking groups that can be used to affect the solubility of the targeting agent and/or that function to attach the cleavable linker to the antibody component and the targeting agent.

The cleavable moiety can comprise any number of functional groups. For example, the cleavable moiety can comprise a functional group that can be cleaved by a selected cleaving agent. As another example, the cleavable moiety can comprise a functional group that can be cleaved under selected cleaving conditions, or by a selected chemical reaction. Thus, cleavable moieties can include functional groups that can be photolytically, chemically, thermally, or enzymatically cleaved. See, e.g., U.S. Pat. No. 5,721,099; U.S. Patent Publication No. 20040166529; and Greene et al., PROTECTIVE GROUPS IN ORGANIC SYNTHESIS, 2nd ed. Wiley, 1991. For example, U.S. Patent Publication No. 20050255042 describes cleavable linking groups that can be enzymatically cleaved by a protease.

### VII. Methods of Administration

The antibody conjugates of the present invention have particular utility in human and veterinary imaging, therapeutic, and diagnostic applications. For example, the antibody conjugates can be used for imaging tumors and for treating diseases and disorders such as cancer, inflammatory diseases, autoimmune diseases, infectious diseases, and neurological disorders.

Administration of the antibody conjugates of the present invention with a suitable pharmaceutical excipient as necessary can be carried out via any of the accepted modes of administration. Thus, administration can be, for example, intravenous, topical, subcutaneous, transcutaneous, transdermal, intramuscular, oral, intra-joint, parenteral, intra-arteriole, intradermal, intraventricular, intracranial, intraperitoneal, intralesional, intranasal, rectal, vaginal, or by inhalation. Moreover, where injection is to treat a tumor, administration may be directly to the tumor and/or into tissues surrounding the tumor.

The compositions containing an antibody conjugate or a combination of antibody conjugates of the present invention may be administered repeatedly, e.g., at least 2, 3, 4, 5, 6, 7, 8, or more times, or the composition may be administered by continuous infusion. Suitable sites of administration include, but are not limited to, dermal, mucosal, bronchial, gastrointestinal, anal, vaginal, eye, and ear. The formulations may take the form of solid, semi-solid, lyophilized powder, or liquid dosage forms, such as, for example, tablets, pills, lozenges, capsules, powders, solutions, suspensions, emulsions, suppositories, retention enemas, creams, ointments, lotions, gels, aerosols, or the like, preferably in unit dosage forms suitable for simple administration of precise dosages.

The term “unit dosage form” refers to physically discrete units suitable as unitary dosages for human subjects and other mammals (e.g., dogs), each unit containing a predetermined quantity of active material calculated to produce the desired onset, tolerability, and/or therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of an antibody conjugate or a combination of antibody conjugates.

Methods for preparing such dosage forms are known to those skilled in the art (see, for example, REMINGTON'S PHARMACEUTICAL SCIENCES, 18TH ED., Mack Publishing Co., Easton, Pa. (1990)). The composition to be administered contains a quantity of the antibody conjugate or combination of antibody conjugates in a pharmaceutically effective amount for imaging a tumor, organ, or tissue or for relief of a condition being treated, when administered in accordance with the teachings of this invention. In addition, pharmaceutically acceptable salts of the antibody conjugates of the present invention (e.g., acid addition salts) may be prepared and included in the compositions using standard procedures known to those skilled in the art of synthetic organic chemistry and described, e.g., by March, Advanced Organic Chemistry: Reactions, Mechanisms and Structure, 4th Ed., New York, Wiley-Interscience (1992).

The compositions typically include a conventional pharmaceutical carrier or excipient and may additionally include other medicinal agents, carriers, adjuvants, diluents, tissue permeation enhancers, solubilizers, and the like. Preferably, the composition will contain about 0.01% to about 90%, about 0.1% to about 75%, about 0.1% to 50%, or about 0.1% to 10% by weight of an antibody conjugate of the present invention or a combination thereof, with the remainder consisting of suitable pharmaceutical carrier and/or excipients. Appropriate excipients can be tailored to the particular composition and route of administration by methods well known in the art. See, e.g., REMINGTON'S PHARMACEUTICAL SCIENCES, supra.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The compositions can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; coloring agents; and flavoring agents. The compositions may also comprise biodegradable polymer beads, dextran, and cyclodextrin inclusion complexes.

For oral administration, the compositions can be in the form of tablets, lozenges, capsules, emulsions, suspensions, solutions, syrups, sprays, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the pharmaceutical compositions take the form of a pill, tablet, or capsule, and thus, the composition can contain, along with the antibody conjugate or combination of antibody conjugates, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. The antibody conjugates can also be formulated into a suppository disposed, for example, in a polyethylene glycol (PEG) carrier.

Liquid compositions can be prepared by dissolving or dispersing an antibody conjugate or a combination of antibody conjugates and optionally one or more pharmaceutically acceptable adjuvants in a carrier such as, for example, aqueous saline (e.g., 0.9% w/v sodium chloride), aqueous dextrose, glycerol, ethanol, and the like, to form a solution or suspension, e.g., for oral, topical, or intravenous administration. The antibody conjugates of the present invention can also be formulated into a retention enema.

For topical administration, the compositions of the present invention can be in the form of emulsions, lotions, gels, creams, jellies, solutions, suspensions, ointments, and transdermal patches. For delivery by inhalation, the composition can be delivered as a dry powder or in liquid form via a nebulizer. For parenteral administration, the compositions can be in the form of sterile injectable solutions and sterile packaged powders. Preferably, injectable solutions are formulated at a pH of about 4.5 to about 7.5.

The compositions of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

Generally, administered dosages will be effective to deliver picomolar to micromolar concentrations of the antibody conjugate to the appropriate site or sites. However, one of ordinary skill in the art understands that the dose administered will vary depending on a number of factors, including, but not limited to, the particular antibody conjugate or set of antibody conjugates to be administered, the mode of administration, the type of application (e.g., imaging, therapeutic), the age of the patient, and the physical condition of the patient. Preferably, the smallest dose and concentration required to produce the desired result should be used. Dosage should be appropriately adjusted for children, the elderly, debilitated patients, and patients with cardiac and/or liver disease. Further guidance can be obtained from studies known in the art using experimental animal models for evaluating dosage. However, the increased tumor penetration, tumor retention, blood clearance, and tumor to blood ratios associated with the antibody conjugates of the present invention permits a wider margin of safety for dosage concentrations and for repeated dosing.

### VIII. Therapeutic Applications

In certain aspects, the antibody conjugates of the present invention are used for the treatment of a disease or disorder in a subject in need thereof. Examples of diseases or disorders suitable for treatment include, but are not limited to, allergy, anxiety disorder, autoimmune disease, behavioral disorder, birth defect, blood disorder, bone disease, cancer, circulatory disease, tooth disease, depressive disorder, dissociative disorder, ear condition, eating disorder, eye condition, food allergy, food-borne illness, gastrointestinal disease, genetic disorder, heart disease, hormonal disorder, immune deficiency, infectious disease, inflammatory disease, insect-transmitted disease, nutritional disorder, kidney disease, leukodystrophy, liver disease, mental health disorder, metabolic disease, mood disorder, musculodegenerative disorder, neurological disorder, neurodegenerative disorder, neuromuscular disorder, personality disorder, phobia, pregnancy complication, prion disease, prostate disease, psychological disorder, psychiatric disorder, respiratory disease, sexual disorder, skin condition, sleep disorder, speech-language disorder, sports injury, tropical disease, vestibular disorder, and wasting disease.

Cancer generally includes any of various malignant neoplasms characterized by the proliferation of anaplastic cells that tend to invade surrounding tissue and metastasize to new body sites. Non-limiting examples of different types of cancer suitable for treatment using the antibody conjugates of the present invention include ovarian cancer, breast cancer, lung cancer, bladder cancer, thyroid cancer, liver cancer, pleural cancer, pancreatic cancer, cervical cancer, prostate cancer, testicular cancer, colon cancer, anal cancer, bile duct cancer, gastrointestinal carcinoid tumors, esophageal cancer, gall bladder cancer, rectal cancer, appendix cancer, small intestine cancer, stomach (gastric) cancer, renal cancer (i.e., renal cell carcinoma), cancer of the central nervous system, skin cancer, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

One skilled in the art will also appreciate that the antibody conjugates of the present invention can be co-administered with other therapeutic agents for the treatment of cancer. Suitable anti-cancer agents for combination therapy include, without limitation, cytotoxins and agents such as antimetabolites, alkylating agents, anthracyclines, antibiotics, antimitotic agents, procarbazine, hydroxyurea, asparaginase, corticosteroids, interferons, radiopharmaceuticals, peptides with anti-tumor activity such as TNF-α, pharmaceutically acceptable salts thereof, derivatives thereof, prodrugs thereof, and combinations thereof.

Inflammatory diseases typically include diseases or disorders characterized or caused by inflammation. Inflammation can result from a local response to cellular injury that is marked by capillary dilatation, leukocytic infiltration, redness, heat, and pain that serves as a mechanism initiating the elimination of noxious agents and damaged tissue. The site of inflammation can include, for example, the lungs, the pleura, a tendon, a lymph node or gland, the uvula, the vagina, the brain, the spinal cord, nasal and pharyngeal mucous membranes, a muscle, the skin, bone or bony tissue, a joint, the urinary bladder, the retina, the cervix of the uterus, the canthus, the intestinal tract, the vertebrae, the rectum, the anus, a bursa, a follicle, and the like. Examples of inflammatory diseases suitable for treatment using the antibody conjugates of the present invention include, but are not limited to, inflammatory bowel disease (e.g., Crohn's disease or ulcerative colitis), rheumatoid diseases such as rheumatoid arthritis, fibrositis, pelvic inflammatory disease, acne, psoriasis, actinomycosis, dysentery, biliary cirrhosis, Lyme disease, heat rash, Stevens-Johnson syndrome, mumps, pemphigus vulgaris, and blastomycosis.

Autoimmune diseases generally include diseases or disorders resulting from an immune response against a self-tissue or tissue component such as, e.g., a self-antibody response or cell-mediated response. Examples of autoimmune diseases suitable for treatment using the antibody conjugates of the present invention include, without limitation, organ-specific autoimmune diseases, in which an autoimmune response is directed against a single tissue, such as Type I diabetes mellitus, myasthenia gravis, vitiligo, Graves' disease, Hashimoto's disease, Addison's disease, autoimmune gastritis, and autoimmune hepatitis; and non-organ specific autoimmune diseases, in which an autoimmune response is directed against a component present in several or many organs throughout the body, such as systemic lupus erythematosus, progressive systemic sclerosis and variants, polymyositis, and dermatomyositis. Additional autoimmune diseases include, for example, pernicious anemia, primary biliary cirrhosis, autoimmune thrombocytopenia, Sjögren's syndrome, and multiple sclerosis.

One skilled in the art will appreciate that the antibody conjugates of the present invention can be co-administered with other therapeutic agents for the treatment of inflammatory or autoimmune diseases. Suitable anti-inflammatory agents for combination therapy include, without limitation, corticosteroids, non-steroidal anti-inflammatory agents, antibodies such as infliximab, 5-aminosalicylates, antibiotics, pharmaceutically acceptable salts thereof; derivatives thereof, prodrugs thereof, and combinations thereof. Suitable immunosuppressive agents for combination therapy include, without limitation, azathioprine and metabolites thereof, anti-metabolites such as methotrexate, immunosuppressive antibodies, mizoribine monophosphate, cyclosporine, scoparone, FK-506 (tacrolimus), FK-778, rapamycin (sirolimus), glatiramer acetate, mycopehnolate, pharmaceutically acceptable salts thereof, derivatives thereof, prodrugs thereof, and combinations thereof.

In another embodiment, the antibody conjugates of the present invention are useful for treating an infection or infectious disease caused by, e.g., a virus, bacterium, fungus, parasite, or any other infectious agent. Non-limiting examples of infectious diseases suitable for treatment include, but are not limited to, acquired immunodeficiency syndrome (AIDS/HIV) or HIV-related disorders, Alpers syndrome, anthrax, bovine spongiform encephalopathy (mad cow disease), chicken pox, cholera, conjunctivitis, Creutzfeldt-Jakob disease (CJD), dengue fever, Ebola, elephantiasis, encephalitis, fatal familial insomnia, Fifth's disease, Gerstmann-Straussler-Scheinker syndrome, hantavirus, helicobacter pylori, hepatitis (hepatitis A, hepatitis B, hepatitis C), herpes, influenza (e.g., avian influenza A (bird flu)), Kuru, leprosy, lyme disease, malaria, hemorrhagic fever (e.g., Rift Valley fever, Crimean-Congo hemorrhagic fever, Lassa fever, Marburg virus disease, and Ebola hemorrhagic fever), measles, meningitis (viral, bacterial), mononucleosis, nosocomial infections, otitis media, pelvic inflammatory disease (PID), plague, pneumonia, polio, prion disease, rabies, rheumatic fever, roseola, Ross River virus infection, rubella, salmonellosis, septic arthritis, sexually transmitted diseases (STDs), shingles, smallpox, strep throat, tetanus, toxic shock syndrome, toxoplasmosis, trachoma, tuberculosis, tularemia, typhoid fever, valley fever, whooping cough, and yellow fever.

In certain embodiments, the antibody conjugates of the present invention are useful for treating a neurological or musculoskeletal disorder. Examples of such disorders include, but are not limited to, Alzheimer's disease, Aicardi syndrome, amnesia, amyotrophic lateral sclerosis (Lou Gehrig's Disease), anencephaly, aphasia, arachnoiditis, Arnold Chiari malformation, ataxia telangiectasia, Batten disease, Bell's palsy, brachial plexus injury, brain injury, brain tumor, Charcol-Marie-Tooth disease, encephalitis, epilepsy, essential tremor, Guillain-Barre Syndrome, hydrocephalus, hyperhidrosis, Krabbes disease, meningitis, Moebius syndrome, muscular dystrophy, multiple sclerosis, Parkinson's disease, peripheral neuropathy, postural or orthostatic tachycardia syndrome, progressive supranuclear palsy, Reye's syndrome, shingles, Shy-Drager Syndrome, spasmodic torticollis, spina bifida, spinal muscular atrophy, Stiff Man syndrome, synesthesia, syringomyelia, thoracic outlet syndrome, Tourette syndrome, toxoplasmosis, and trigeminal neuralgia.

The antibody conjugates described herein are also useful for the treatment of conditions associated with exposure to toxins (e.g., snake venom, bacterial toxins, other neurotoxins, etc.) or drugs (e.g., drug abuse, drug overdose, alcoholism, etc.). For example, an antibody conjugate or combination of antibody conjugates specific for a particular toxin or drug can be administered to a subject as an antidote for the toxin or drug. In this manner, the antibody conjugates of the present invention can be used as anti-venom agents for the treatment of, e.g., snake bites, botulism, or tetanus, or as detoxifying agents for the treatment of, e.g., drug overdoses or poisoning.

### IX. Imaging Applications

In certain other aspects, the antibody conjugates of the present invention are used as in vivo optical imaging agents of tissues and organs in various biomedical applications including, but not limited to, imaging of tumors, tomographic imaging of organs, monitoring of organ functions, coronary angiography, fluorescence endoscopy, laser guided surgery, photoacoustic and sonofluorescence methods, and the like. In one embodiment, the antibody conjugates of the invention are useful for the detection of the presence of tumors and other abnormalities by monitoring where a particular antibody conjugate is concentrated in a subject. In another embodiment, the antibody conjugates are useful for laser-assisted guided surgery for the detection of micro-metastases of tumors upon laparoscopy. In yet another embodiment, the antibody conjugates are useful in the diagnosis of atherosclerotic plaques and blood clots.

In further embodiments, the antibody conjugates of the present invention are used in the imaging of: (1) ocular diseases in ophthalmology, e.g., to enhance the visualization of chorioretinal diseases such as vascular disorders, retinopathies, neovascularization, and tumors via direct microscopic imaging; (2) skin diseases such as skin tumors via direct microscopic imaging; (3) gastrointestinal, oral, bronchial, cervical, and urinary diseases and tumors via endoscopy; (4) atherosclerotic plaques and other vascular abnormalities via flexible endocsopic catheters; (5) breast tumors via 2D- or 3D-image reconstruction; and (6) brain tumors, perfusion, and stroke via 2D- or 3D-image reconstruction.

The antibody conjugates of the present invention can be administered either systemically or locally to the tumor, organ, or tissue to be imaged, prior to the imaging procedure. Generally, the antibody conjugates are administered in doses effective to achieve the desired optical image of a tumor, tissue, or organ. Such doses may vary widely, depending upon the particular antibody conjugate employed, the tumor, tissue, or organ subjected to the imaging procedure, the imaging equipment being used, and the like.

In some embodiments, the antibody conjugates described herein are used to directly stain or label a sample so that the sample can be identified or quantitated. For instance, a specific antibody conjugate can be added as part of an assay for a biological target analyte (e.g., antigen), as a detectable tracer element in a biological or non-biological fluid, or for other in vitro purposes known to one of skill in the art. Typically, the sample is obtained directly from a liquid source or as a wash from a solid material (organic or inorganic) or a growth medium in which cells have been introduced for culturing, or a buffer solution in which cells have been placed for evaluation. Where the sample comprises cells, the cells are optionally single cells, including microorganisms, or multiple cells associated with other cells in two or three dimensional layers, including multicellular organisms, embryos, tissues, biopsies, filaments, biofilms, and the like.

A detectable response generally refers to a change in, or occurrence of, an optical signal that is detectable either by observation or instrumentally. In certain instances, the detectable response is radioactivity (i.e., radiation), including alpha particles, beta particles, nucleons, electrons, positrons, neutrinos, and gamma rays emitted by a radioactive substance such as a radionuclide. In certain other instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ).

When used in imaging applications, the antibody conjugates of the present invention typically have an imaging agent covalently or noncovalently attached thereto. Suitable imaging agents include, but are not limited to, radionuclides, detectable tags, fluorophores, fluorescent proteins, enzymatic proteins, and the like. One of skill in the art will understand that the above-described methods for attaching targeting agents to antibodies or antibody fragments can also be used for attaching imaging agents to antibody conjugates. In addition, one of skill in the art will be familiar with other methods for attaching imaging agents to antibody conjugates. For example, the imaging agent can be attached to the antibody portion of the antibody conjugate via site-specific conjugation, e.g., covalent attachment of the imaging agent to a peptide linker such as a polyarginine moiety having five to seven arginines present at the carboxyl-terminus of the antibody molecule. The imaging agent can also be directly attached to the targeting agent and/or antibody portion of the antibody conjugate via non-site specific conjugation, e.g., covalent attachment of the imaging agent to primary amine groups present in the constant (CH) domains of the antibody heavy chain. One of skill in the art will appreciate that an imaging agent can also be bound to an antibody conjugate via noncovalent interactions (e.g., ionic bonds, hydrophobic interactions, hydrogen bonds, Van der Waals forces, dipole-dipole bonds, etc.).

In certain instances, the antibody conjugate is radiolabeled with a radionuclide by directly attaching the radionuclide to the targeting agent and/or antibody portion of the antibody conjugate. In certain other instances, the radionuclide is bound to a chelating agent or chelating agent-linker attached to the antibody conjugate Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Preferably, the radionuclide bound to a chelating agent is ⁶⁴Cu, ⁹⁰Y, ¹¹¹In, or mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to the antibody conjugates of the present invention. In particular, attachment can be conveniently accomplished using, for example, commercially available bifunctional linking groups (generally heterobifunctional linking groups) that can be attached to a functional group present in a non-interfering position on the antibody conjugate and then further linked to a radionuclide, chelating agent, or chelating agent-linker.

Non-limiting examples of fluorophores or fluorescent dyes suitable for use as imaging agents include Alexa Fluor® dyes (Invitrogen Corp.; Carlsbad, Calif.), fluorescein, fluorescein isothiocyanate (FITC), Oregon Green™; rhodamine, Texas red, tetrarhodamine isothiocynate (TRITC), CyDye™ fluors (e.g., Cy2, Cy3, Cy5), and the like.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al., Mol. Microbiol., 55:1767-1781 (2005), the GFP variant described in Crameri et al., Nat. Biotechnol., 14:315-319 (1996), the cerulean fluorescent proteins described in Rizzo et al., Nat. Biotechnol, 22:445 (2004) and Tsien, Annu. Rev. Biochem., 67:509 (1998), and the yellow fluorescent protein described in Nagal et al., Nat. Biotechnol., 20:87-90 (2002). DsRed variants are described in, e.g., Shaner et al., Nat. Biotechnol., 22:1567-1572 (2004), and include mStrawberry, mCherry, morange, mBanana, mHoneydew, and mTangerine. Additional DsRed variants are described in, e.g., Wang et al., Proc. Natl. Acad. Sci. U.S.A., 101:16745-16749 (2004) and include mRaspberry and mPlum. Further examples of DsRed variants include mRFPmars described in Fischer et al., FEBS Lett., 577:227-232 (2004) and mRFPruby described in Fischer et al., FEBS Lett., 580:2495-2502 (2006).

In other embodiments, the imaging agent that is bound to an antibody conjugate of the present invention comprises a detectable tag such as, for example, biotin, avidin, streptavidin, or neutravidin. In further embodiments, the imaging agent comprises an enzymatic protein including, but not limited to, luciferase, chloramphenicol acetyltransferase, β-galactosidase, β-glucuronidase, horseradish peroxidase, xylanase, alkaline phosphatase, and the like.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled antibody conjugate of the present invention. Positron emission tomography (PET) is another suitable technique for detecting radiation in a subject. Furthermore, U.S. Pat. No. 5,429,133 describes a laparoscopic probe for detecting radiation concentrated in solid tissue tumors. Miniature and flexible radiation detectors intended for medical use are produced by Intra-Medical LLC (Santa Monica, Calif.). Magnetic Resonance Imaging (MRI) or any other imaging technique known to one of skill in the art is also suitable for detecting the radioactive emissions of radionuclides. Regardless of the method or device used, such detection is aimed at determining where the antibody conjugate is concentrated in a subject, with such concentration being an indicator of the location of a tumor or tumor cells.

Non-invasive fluorescence imaging of animals and humans can also provide in vivo diagnostic information and be used in a wide variety of clinical specialties. For instance, techniques have been developed over the years for simple ocular observations following UV excitation to sophisticated spectroscopic imaging using advanced equipment (see, e.g., Andersson-Engels et al., Phys. Med. Biol., 42:815-824 (1997)). Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol., 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al., IEEE Transactions on Biomedical Engineering, 48:1034-1041 (2001), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

### X. Kits of the Invention

The present invention also provides kits to facilitate and/or standardize the use of the compositions provided herein, as well as to facilitate the methods described herein. Materials and reagents to carry out these various methods can be provided in kits to facilitate execution of the methods. As used herein, the term “kit” includes a combination of articles that facilitates a process, assay, analysis, or manipulation. In particular, kits comprising the antibody conjugates of the present invention find utility in a wide range of applications including, for example, immunotherapy and in vivo imaging a cell, tumor, organ, tissue, bioaggregate, biofilm, or the like.

Kits can contain chemical reagents (e.g., chemical antibodies) as well as other components. In addition, the kits of the present invention can include, without limitation, instructions to the kit user (e.g., directions for use of the antibody conjugate in immunotherapy, directions for use of the antibody conjugate in imaging a cell, tumor, organ, or tissue, etc.), apparatus and reagents for sample collection and/or purification, apparatus and reagents for product collection and/or purification, reagents for bacterial cell transformation, reagents for eukaryotic cell transfection, previously transformed or transfected host cells, sample tubes, holders, trays, racks, dishes, plates, solutions, buffers or other chemical reagents, suitable samples to be used for standardization, normalization, and/or control samples. Kits of the present invention can also be packaged for convenient storage and safe shipping, for example, in a box having a lid.

### XI. Examples

The following examples are offered to illustrate, but not to limit, the claimed invention.

### Example 1

### Synthesis and Characterization of Antibody Conjugates

This example illustrates the synthesis and characterization of stable, intermediate-sized antibody conjugates produced by the non-site specific conjugation of peptidomimetic ligands specific for the α₄β₁ integrin receptor (e.g., Ligand 2A (also known as LLP2A), Ligand 1A, etc.) to the Fc portion of human IgG1. This examples also illustrates that the synthesized antibody conjugates are capable of binding to target cells and inducing antibody-dependent cell-mediated cytotoxicity (ADCC).

### Solid Phase Synthesis of Ligand 2A-DOPA-Biotin and Other Ligands

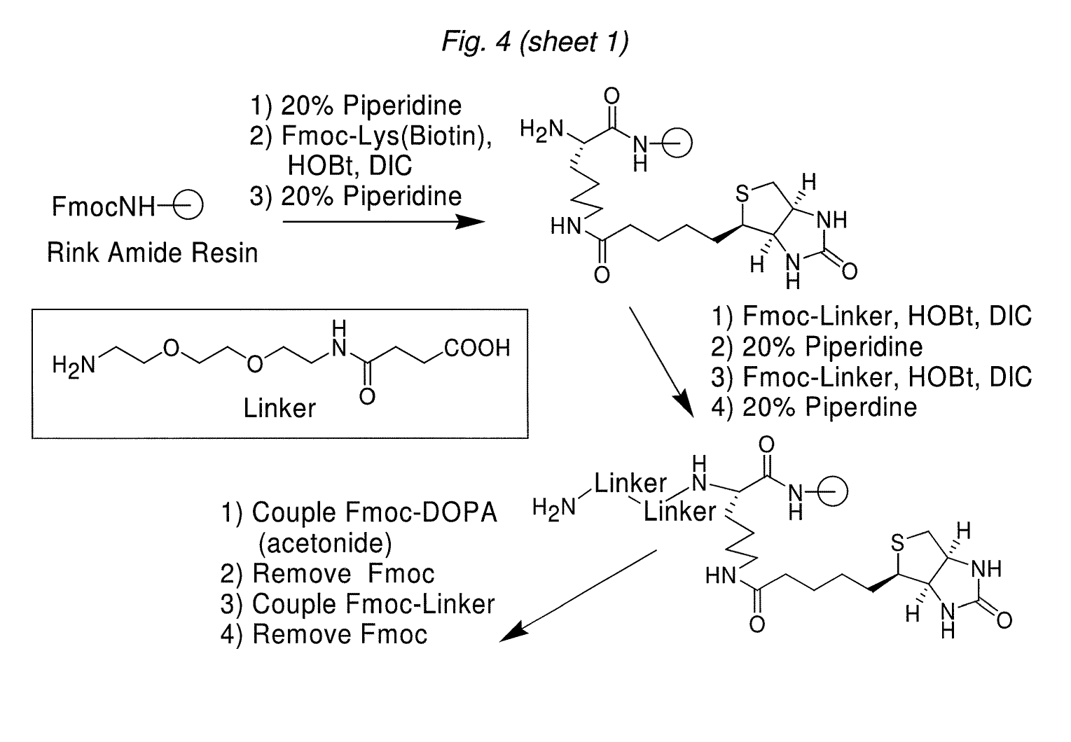

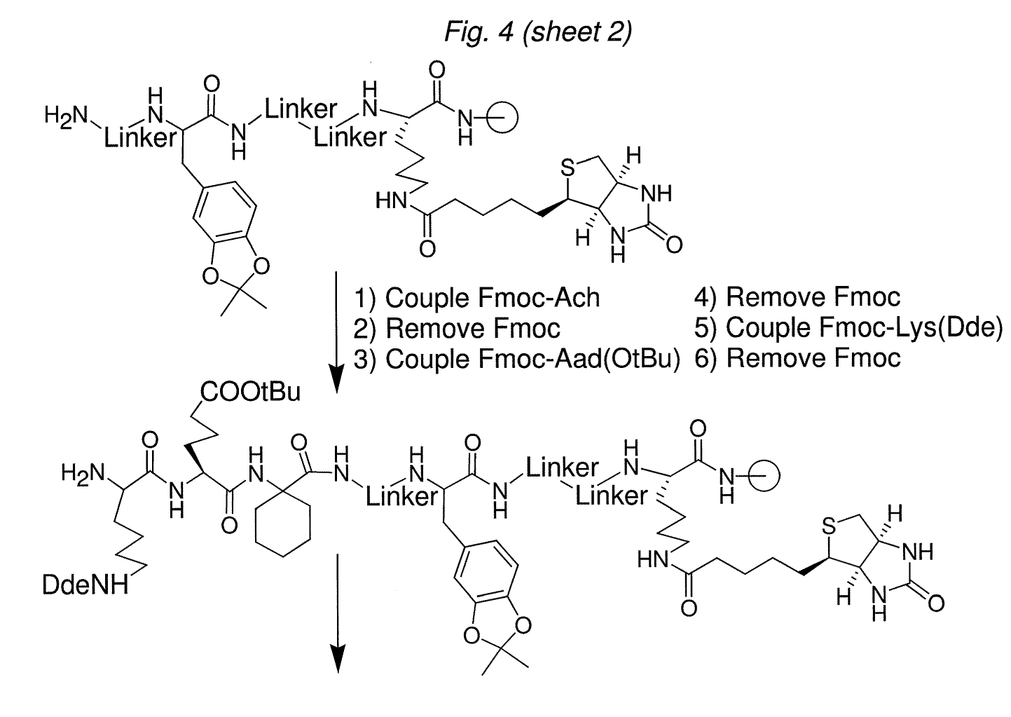

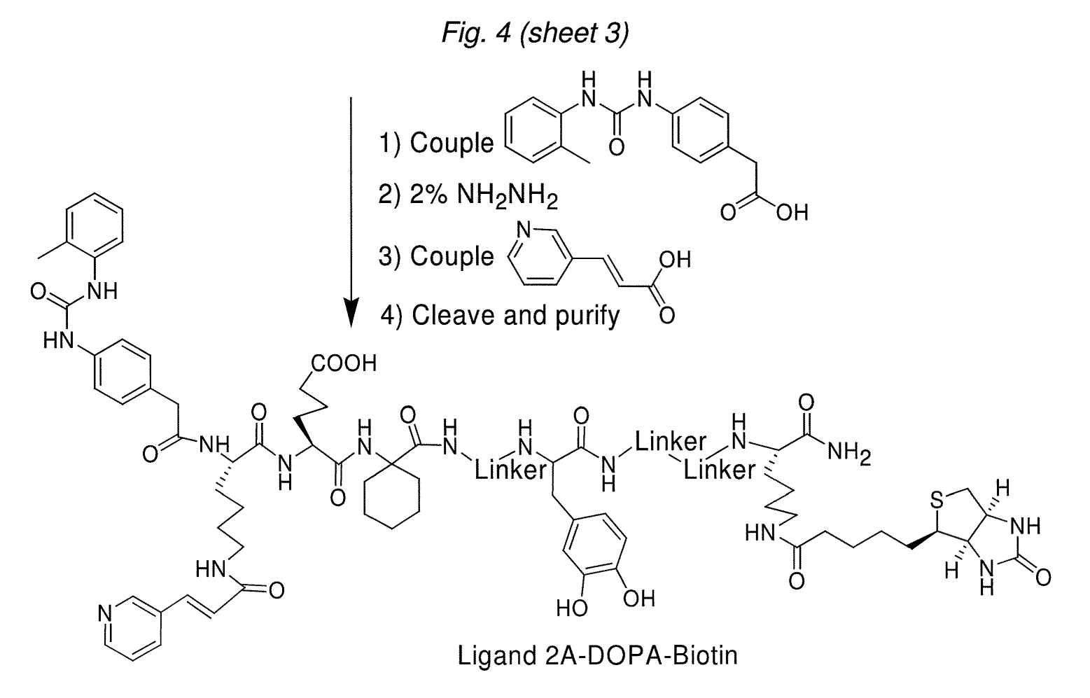

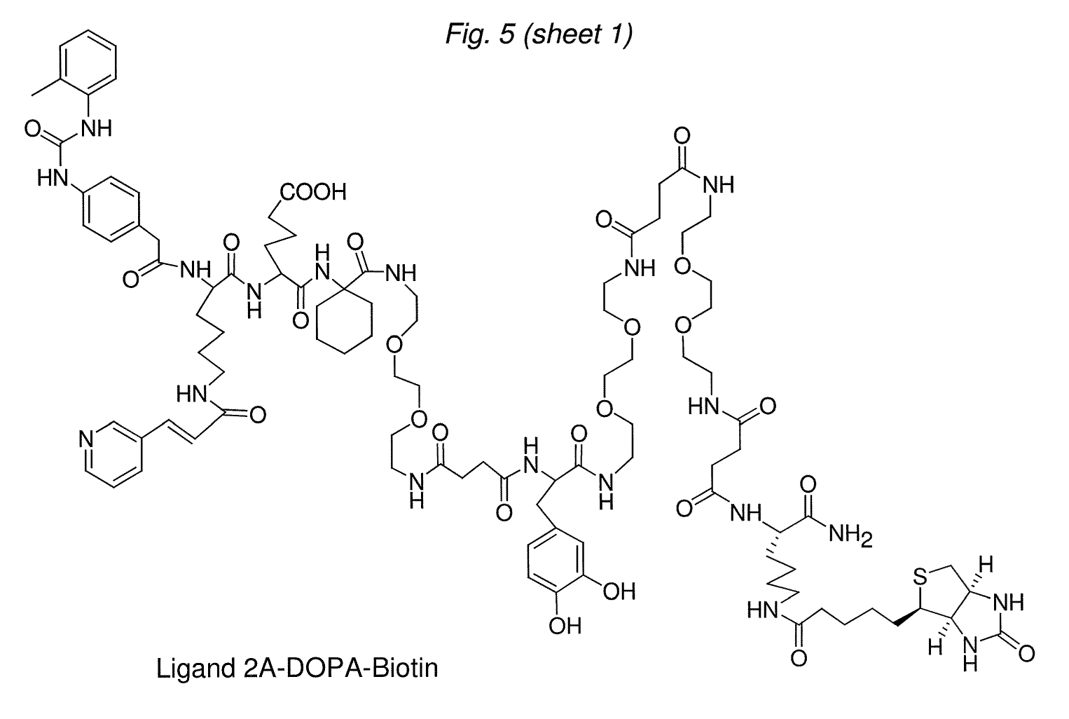

The synthetic scheme for Ligand 2A-DOPA-Biotin is shown in FIG. 4. The 3,4-dihydroxy-phenylalanine (DOPA) moiety permits amine-coupling of the ligand to the IgG1 Fc fragment in the presence of an oxidizing agent such as sodium periodate. Biotin was also incorporated into the ligand as a means to monitor ligand conjugation to the IgG1 Fc fragment and to visualize cell staining.

Rink amide MBHA resin (0.5 g, 0.325 mmol, loading 0.65 mmol/g) was swollen in DMF for 3 h before Fmoc-deprotection with 20% piperidine in DMF twice (5 min, 15 min). The beads were washed with DMF, MeOH, and DMF. Fmoc-Lys(Biotin) (0.580 g, 0.975 mmol) was dissolved in a solution of HOBt (0.149 g, 0.975 mmol) and DIC (152 μl, 0.975 mmol) in DMF (8 ml), and was then added into the beads. The coupling was carried out at room temperature overnight. After filtration, the beads were washed with DMF, MeOH, and DMF, respectively, three times each. After removal of Fmoc, the beads were then subjected to two cycles of coupling and deprotection of the Fmoc-linker. Fmoc-DOPA(acetonide) was then coupled to the beads, followed by coupling of the Fmoc-linker.

Ligand 2A was assembled on the beads as follows: Fmoc-Ach-OH (0.365 g, 0.975 mmol) was dissolved in a solution of HOBt (0.149 g, 0.975 mmol) and DIC (152 μl, 0.975 mmol) in DMF, and was then added to the above-mentioned beads. The coupling was carried out at room temperature for 2 h. After filtration, the beads were washed with DMF (3×10 ml), MeOH (3×10 ml), and DMF (3×10 ml), respectively, three times each. The Fmoc deprotection group was removed with 20% piperidine twice (5 min, 15 min). After washing with DMF, MeOH, and DMF respectively, the beads were then subjected to additional coupling and deprotection cycles stepwise with Fmoc-Aad(tBu) and Fmoc-Lys(Dde) in the same manner as described above. After removal of Fmoc, a solution of 4-[(N′-2-methylphenyl)ureido]phenylacetic acid (0.923 g, 3.25 mmol), HOBt (0.498 g, 3.25 mmol), and DIC (509 μl, 3.25 mmol) in DMF was added to the beads. The reaction was conducted at room temperature overnight. The beads were washed with DMF (3×10 ml), methanol (3×10 ml), and DMF (3×10 ml). The Dde protecting group was removed with 2% NH₂NH₂ in DMF twice (5 min, 10 min). After washing with DMF (3×5 ml), MeOH (3×15 ml), and DMF (3×5 ml), a solution of trans-3-(3-pyridyl)acrylic acid (0.485 g, 3.25 mmol), HOBt (0.498 g, 3.25 mmol), and DIC (509 μl, 3.25 mmol) in DMF (8 ml) was added to the beads. The coupling proceeded at room temperature overnight. A Kaiser test was negative. The beads were washed with DMF (5×5 ml), MeOH (3×5 ml), and DCM (3×5 ml). The beads were then dried in vacuo for 1 h before adding a cleavage mixture of 95% TFA:2.5% water:2.5% TIS. The cleavage reaction was conducted at room temperature for 2 h. The liquid was collected and concentrated. The crude product was precipitated with diethyl ether and purified using preparative RP-HPLC to give Ligand 2A-DOPA-Biotin. MALDI-TOF MS: 2034 (MH⁺).

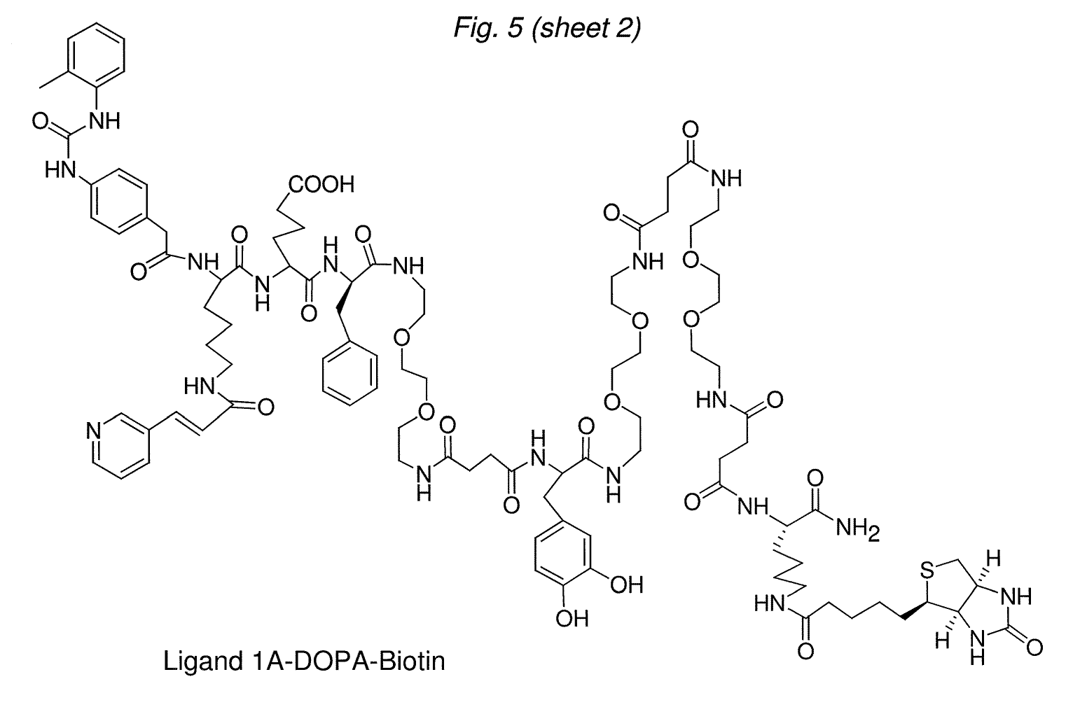

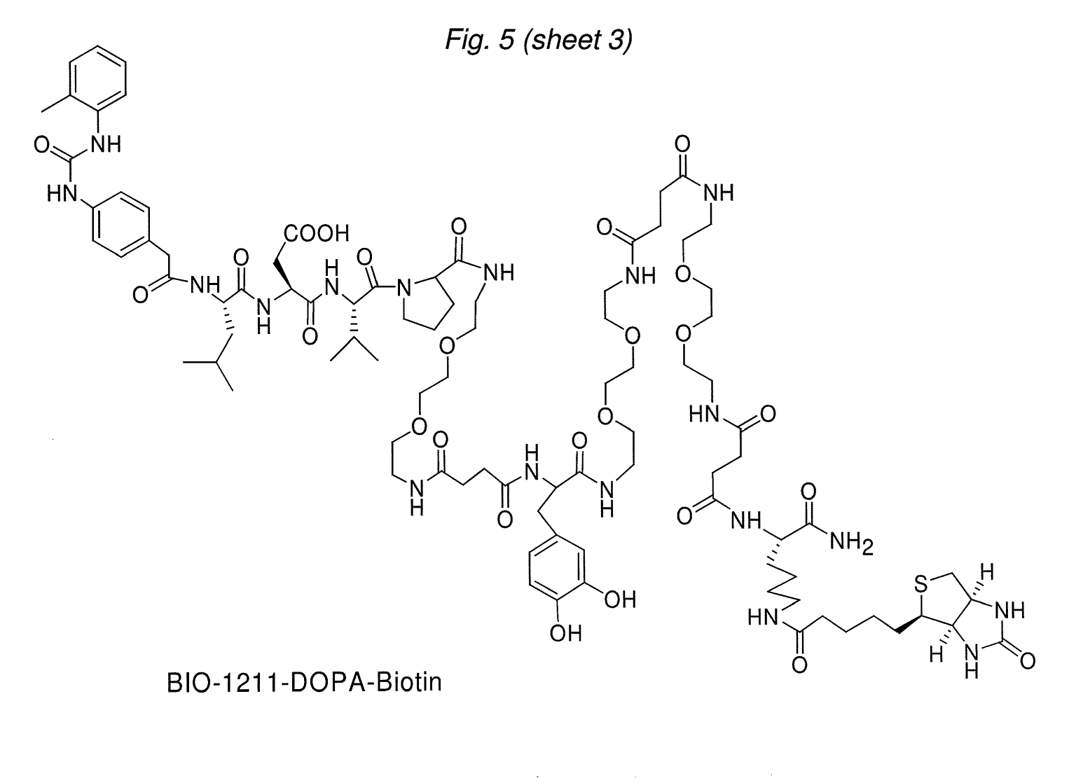

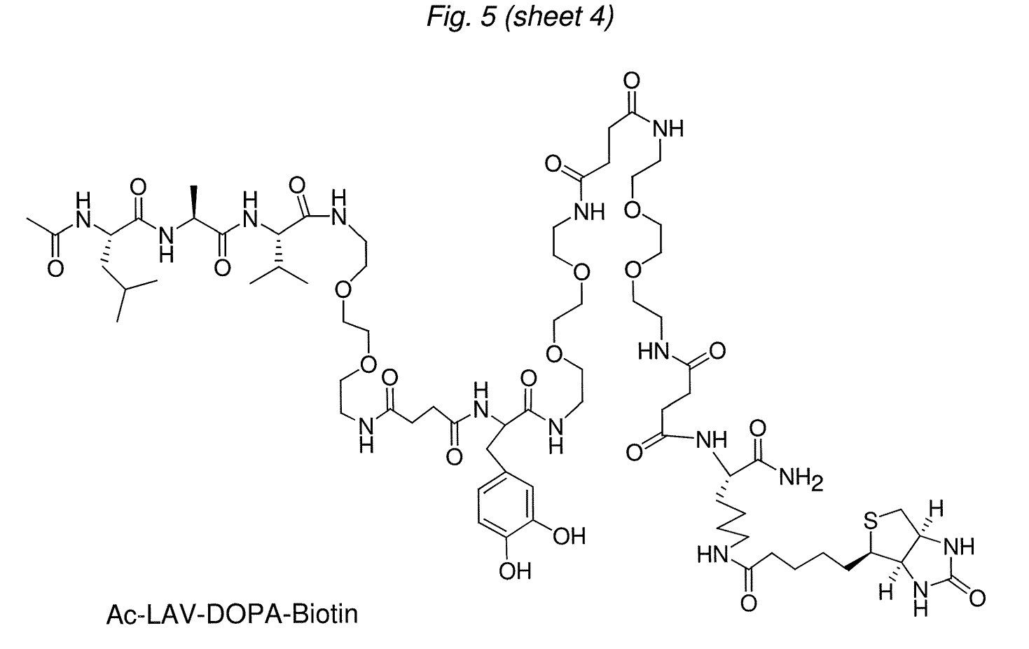

The synthesis of Ligand 1A-DOPA-Biotin, BIO-1211-DOPA-Biotin, andAc-LAV-DOPA-Biotin (negative control) was similar to that of Ligand 2A-DOPA-Biotin, but used different Fmoc-amino acids as building blocks. FIG. 5 shows the chemical structures of each of these ligands. MALDI-TOF MS analysis confirmed their structures.

### Formation of Fc-Ligand Antibody Conjugates

Fc fragments of IgG were first dialyzed against potassium free PBS (3 changes×500 ml, 4° C.). Oxidation of DOPA was achieved using 100 mM sodium periodate in acetonitrile. This solution was sonicated for 10 minutes to ensure saturation. Briefly, the ligand was first dissolved in 100% DMSO. 100 mM NaIO₄ (saturated solution) was added to the ligand for 45 minutes at room temperature. The activated ligand was directly added at a 4:1 molar ratio to the dialyzed antibody Fc fragment and placed on a rotator at room temperature for 3 hours. To remove excess salt, organic solvent, and any unconjugated ligand, the Fc-ligand antibody conjugates were extensively dialyzed against potassium free PBS (4 changes×1 L, 4° C.). A 10K cut-off dialysis chamber was used. The resulting product was a stable, intermediate-sized Fc-ligand antibody conjugate having a molecular weight of about 50-60 kDa.

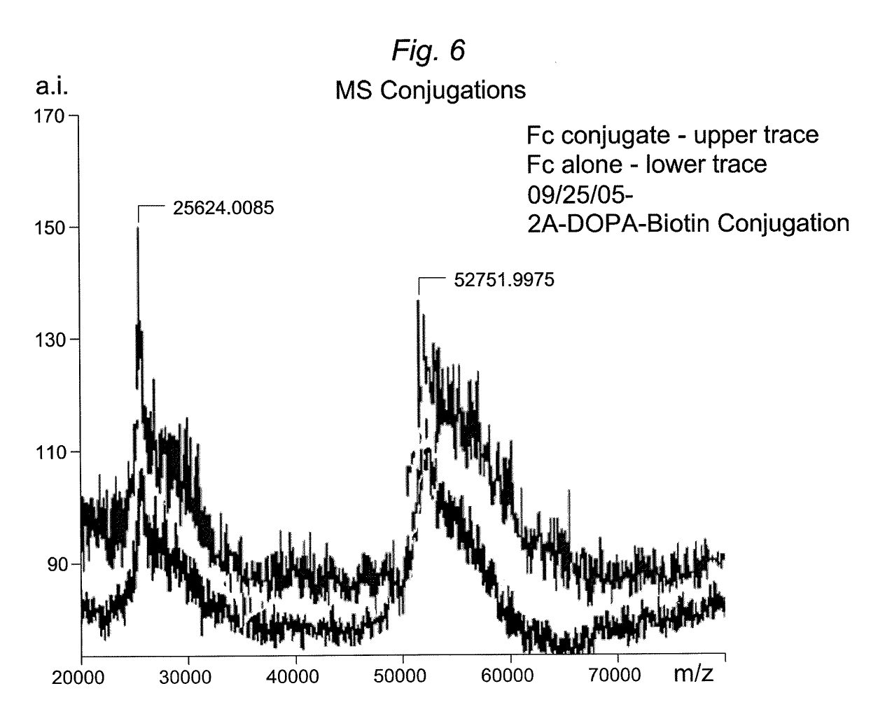

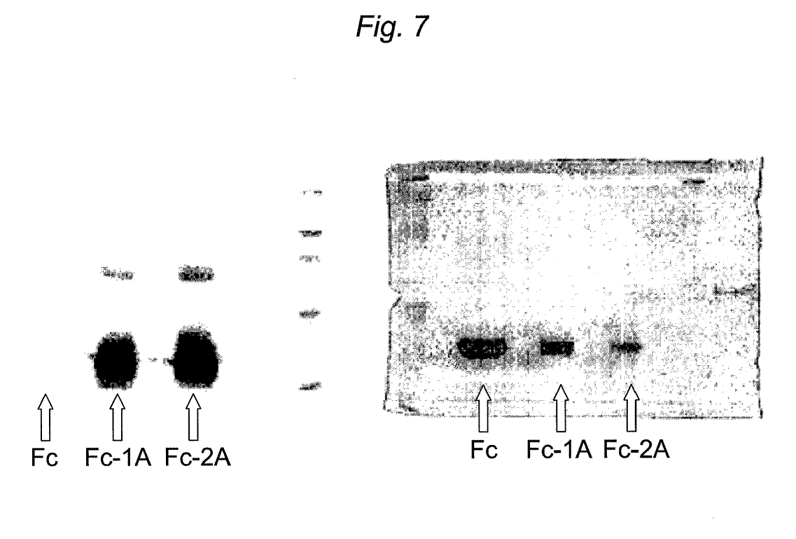

To assess conjugation, Fc-ligand antibody conjugates were analyzed by MALDI-TOF mass spectrometry (MS). As shown in FIG. 6, MS analysis on a Bruker Biflex III indicated that there were two ligands incorporated per Fc fragment. For confirmation, Fc-ligand antibody conjugates were electrophoresed on a 10% SDS gel and transferred to a PVDF membrane for Western blotting. Western blots were then performed using 1:200,000 neutravidin-AP or 1:1000 anti-human Fc (Rockland Immunochemicals, Inc.; Gilbertsville, Pa.) and 1:1000 anti-goat-HRP (Sigma-Aldrich Co.; St. Louis, Mo.) for 1 h each. FIG. 7 shows the detection of Fc-ligand antibody conjugates following Western blot analysis, indicating that Ligands 1A and 2A were conjugated to the Fc fragment, whereas no signal was detected for unconjugated Fc. SDS and Western blot analysis further demonstrated that the conjugation was covalent. The lack of a band shift following SDS gel electrophoresis supported the results obtained using MS.

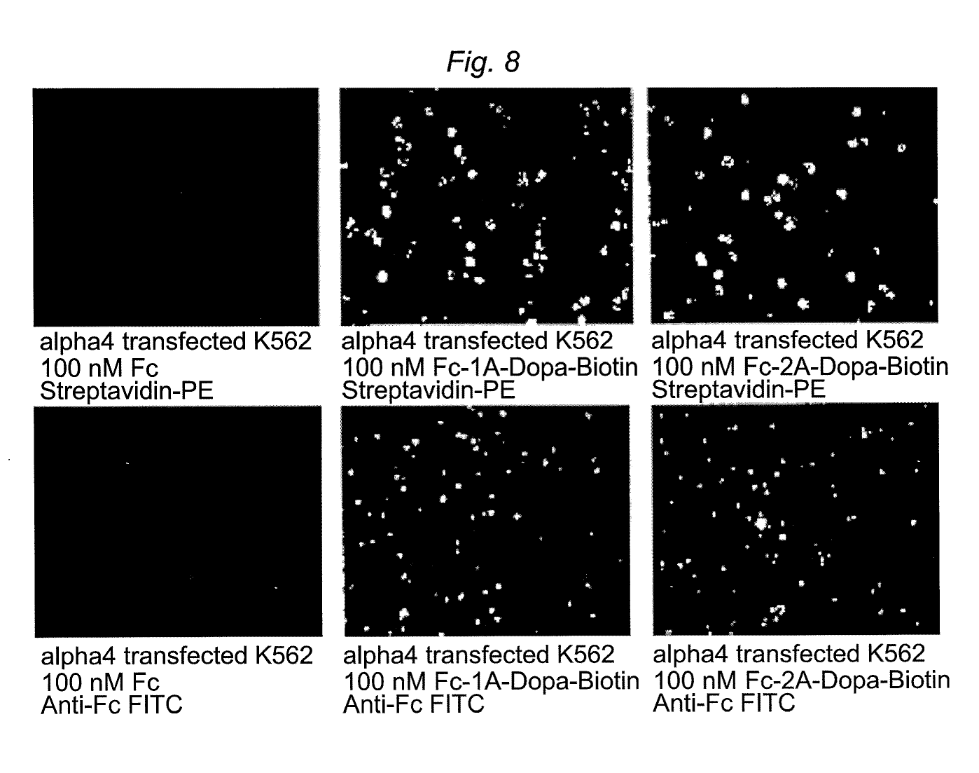

To assess whether the Fc-ligand antibody conjugates were able to bind cells and to assess any loss of reactivity due to steric hindrance, standard immunofluorescence was performed on K562 cells transfected with the alpha4 integrin subunit. K562 cells express the betal integrin subunit, but not the alpha4 integrin subunit. Staining was quantified by FLOW cytometry, which was performed on a Coulter® XL-MCL™ Flow Cytometer (Beckman Coulter, Inc.; Fullerton, Calif.). Cells were harvested and adjusted to a concentration of 1×10⁶ cells/ml in TBS plus 1 mM CaCl₂ and MnCl₂. Fc-ligand antibody conjugates, Fc fragments, or ligands were then incubated at various concentrations with cells for 1 hour at room temperature. Cells were centrifuged at 1000 RPM for 5 minutes, liquid was aspirated, and cells were resuspended and spun down again. Cells were then incubated with a detecting agent, e.g., 1:1000 streptavidin-PE or 1:500 anti-human Fc-FITC (Sigma-Aldrich Co.) for 1 h at room temperature. Cells were washed two times as described above and then resuspended in PBS at a concentration of 1×10⁶ cells/ml. Cellular fluorescence was visualized using a fluorescent microscope. Ligand 2A-DOPA-Biotin alone displayed an IC₅₀ of 2 pM. As shown in FIG. 8, the Fc-Ligand 2A-DOPA-Biotin antibody conjugate or the Fc-Ligand 1A-DOPA-Biotin antibody conjugate at nanomolar concentrations was capable of binding to K562 cells transfected with the alpha4 integrin subunit.

### Assessment of Fc-Ligand Conjugate Bioactivity

To assess whether tumor cells were selectively being targeted and lysed by the Fc-ligand antibody conjugates described herein, the ability of these antibody conjugates to induce antibody-dependent cell-mediated cytotoxicity (ADCC) was determined.

Cells were cultured in a humidified, 5% CO₂ incubator at 37° C. K562 cells (ATCC; Manassas, Va.) and K562 cells transfected with alpha4 integrin were maintained in RPMI medium containing 10% fetal bovine serum. Peripheral blood cells were obtained from normal healthy donors and were collected from Pall RCM1 leukocyte filters. Filters were back-flushed with Hank's Balanced Salt Solution and monocytes were separated using a double density gradient consisting of histopaque-1077 and 1119 (Sigma-Aldrich Co.). Monocyte collection was performed according to the manufacturer's instructions. Following isolation, T cells were depleted by plastic adherence. After a 45 minute incubation, cells not adhering to plastic were removed. NK cells were further enriched using a 5-day treatment with low dose (100 IU/ml) IL-2 (Sigma-Aldrich Co.). Viability was confirmed using trypan blue staining.

To measure cytotoxicity induced by NK cells, the amount of lactose dehydrogenase (LDH) released following cell lysis was measured. K562 or alpha4-transfected K562 tumor target cells (1×10⁴) were plated in 96-well plates with effector cells at a 20:1 effector:target cell ratio. The following compounds were added to each well at a 25 nM concentration: Fc alone; Fc-Ligand 2A-DOPA-Biotin antibody conjugate; or Fc-Ligand 1A-DOPA-Biotin antibody conjugate. Controls for spontaneous lysis of target cells, effector cells, maximum target cell LDH release, and blanks were included as well. After a 4 hour incubation at 37° C. with 5% CO₂, supernatants were collected and LDH release was assayed following the manufacturer's instructions using CytoTox 96 Non-Radioactive Cyotoxicity assay (Promega Corp.; Madison, Wis.). Absorbance was measured at 490 nm. Percent cytotoxicity was calculated using the following formula:

$$\% Cytotoxcity = \frac{\begin{matrix}Experimental - Effector spontaneous release - \\ Target spontaneous release\end{matrix}}{\begin{matrix}Target maximum release - \\ Target spontaneous release\end{matrix}} \times 100.$$

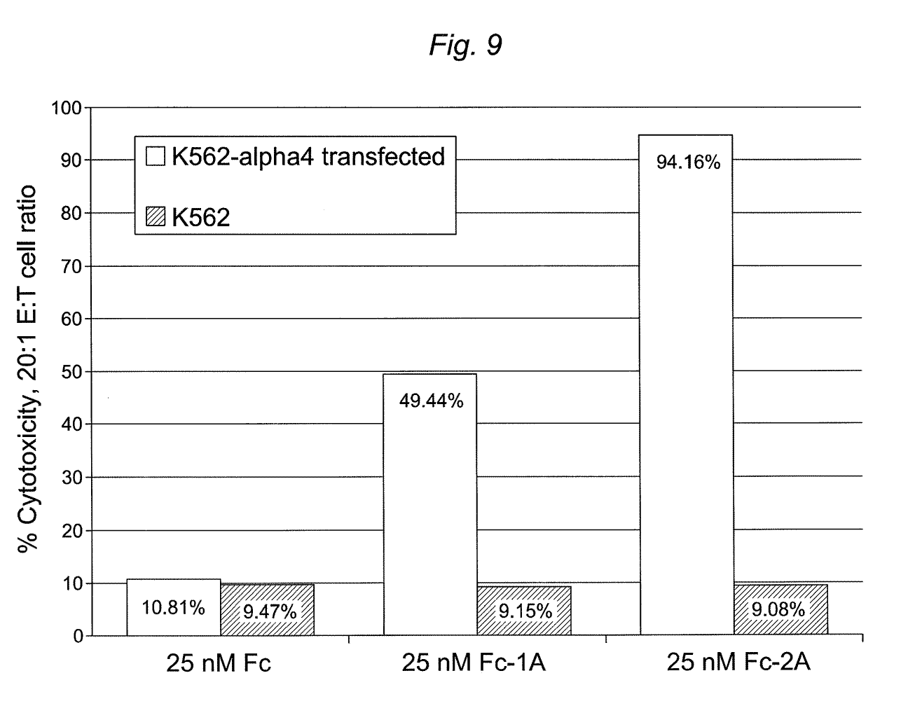

As shown in FIG. 9, a significant level of specific cytotoxicity was demonstrated using the Fc-ligand antibody conjugates described herein. In particular, 94.16% and 49.44% of alpha4-transfected K562 cells lysed in the presence of the Fc-Ligand 2A-DOPA-Biotin antibody conjugate (“Fc-2A”) or the Fc-Ligand 1A-DOPA-Biotin antibody conjugate (“Fc-1A”), respectively, whereas only 10.81% of these cells lysed in the presence of the Fc fragment alone. Conversely, wild-type K562 cells, which express beta-1 integrin but not the alpha-4 subunit, did not lyse in the presence of the Fc-ligand antibody conjugates. Non-specific cell lysis was minimal, i.e., less than 15%.

This example demonstrates that intermediate-sized dimers of antibody heavy chain fragments containing two or more constant domains (e.g., Fc fragments having CH2 and CH3 domains) can be used as an efficient molecular scaffold for delivering chemical ligands such as α₄β₁ integrin ligands to target cells without any loss in biological function.

### Example 2

### Synthesis and Characterization of Additional Antibody Conjugates

This example illustrates the synthesis and characterization of additional antibody conjugates produced by either site-specific or non-site specific conjugation of Ligand 2A (also known as LLP2A) to the Fc portion of human IgG1. This example also illustrates that the synthesized antibody conjugates are capable of binding to target cells and inducing antibody-dependent cell-mediated cytotoxicity (ADCC).

Construction of N-Terminal Modified Human IgG1 Fc Fragments (hIgG1-Cys-Fc)

Conjugation of a targeting agent to one or both N-termini of an antibody heavy chain fragment dimer can be performed in order to preserve the biological properties of both components of the antibody conjugate. As described herein, site-specific conjugation can be conducted using a human IgG1 Fc fragment in which a cysteine amino acid was introduced at the N-terminus of each heavy chain fragment by site-directed mutagenesis (Promega Corp.; Madison, Wis.).

Site-directed mutagenesis: pFUSE-hIgG1-Fc2, a vector expressing a human IgG1 Fc fragment available from InvivoGen (San Diego, Calif.), was used for site-directed mutagenesis to mutate the codon after the signal sequence of the heavy chain fragment to a codon encoding cysteine. Site-directed mutagenesis was performed using the primers shown in Table 2 and the Quick-Change™ site-directed mutagenesis kit (Stratagene; La Jolla, Calif.). The amplified PCR products were digested with DpnI. The digested PCR products were transformed into high efficiency competent cells and bacterial clones were selected on LB medium containing zeocin at 200 μg/ml. Plasmid DNA was isolated from positive clones and sequenced to confirm the mutation. Five clones were selected, and the sequence alignment for one of the clones is shown in Table 2. Plasmid DNA of the mutated clone was used to transfect mammalian CHO (Chinese Hamster Ovary) cells using Lipofectamine-2000. Stable clones expressing a modified form of the IgG1 Fc region having an N-terminal cysteine in both heavy chain fragments (i.e., hIgG1-Cys-Fc) were selected by growing the cells in DMEM medium supplemented with 10% FBS and 500 μg/ml zeomycin.

TABLE 2

| | |
|---|---|
| Primer | Nucleotide sequence 611-653 ofpFUSE-hIgG1-Fc2 |

| | |
|---|---|
| Fo-cys-EP | 5′-CTT GTC ACG AAT TCG TGC TCG GCC ATG GTT AGA TCT GTG GAG-3′ |
| (Forward primer) | |
| Fc-cys-RP | 5′-CTC CAC AGA TCT AAC CAT GGC CGA GCA CGA ATT CGT GAC AAG-3′ |
| (Reverse primer) | |

| | | |
|---|---|---|
| Sequence | pFUSEhIgG1-Fc2 | 605 615 625 |
| Alignment | hIgG1-Cys-Fc | TTGCACTAAG TCTTGCACTT GTCACGAATT |
| | | TTGCACTAAG TCTTGCACTT GTCACGAATT |
| | | 635 645 655 |
| | | CGATATCGGC CATGGTTAGA TCTGACAAAA |
| | | CGTGCTCGGC CATGGTTAGA TCTG------ |

Sequence alignments were performed using Bioedit software. The mutated “Cys” codon is bolded and underlined.

hIgG1-Cys-Fc protein production: The expression level of hIgG1-Cys-Fc was determined by sandwich ELISA. Serum-free supernatants of positive clones were collected and incubated for 1 h in Protein G-coated plates (Pierce Biotechnology; Rockford, Ill.). Supernatants were aspirated and an alkaline phosphatase (AP)-conjugated anti-human Fc-specific antibody (Bethyl, Inc.; Montgomery, Tex.) was added and incubated for 30 min at room temperature. Three PBS washes were performed and DAB substrate was added for color development. Based on the color intensity, highly expressing positive clones were selected for hIgG-Cys-Fc protein production. Serum-free supernatants from highly expressing clones were collected and hIgG1-Cys-Fc protein was purified by Protein G affinity columns (Bio-Rad Laboratories; Hercules, Calif.). The purity of the protein was confirmed by 10% SDS-PAGE under reducing conditions and by transferring the resolved proteins to a nitrocellulose membrane. Another gel was stained with Coomassie blue and the gel pattern was documented. Western blots were developed using AP-conjugated goat anti-human Fc-specifc antibody using s chemiluminescence kit (PerkinElmer Inc.; Waltham, Mass.). A band of about 25 kDa reacted with anti-human IgG Fc-specific antibodies under reducing conditions, which corresponded to the monomeric form of hIgG1-Cys-Fc. As such, the estimated molecular weight of the dimeric hIgG1-Cys-Fc protein is about 50 kDa.

Coniugation of Ligand 2A to hIgG1-Cys-Fc Fragments and hIgG1

Ligand 2A is a targeting ligand specific for the α₄β₁ integrin receptor, which is overexpressed in lymphomas. α₄β₁ integrin plays a major role in facilitating metastatic disease. For instance, α₄β₁ integrin is expressed only on proliferating cells during tumor development, but not on quiescent cells. In B-cell chronic lymphocytic leukemia, α₄β₁ integrin is linked to drug resistance and resistance to apoptosis. In a murine model of myeloma, a monoclonal antibody to α₄β₁ integrin was shown to inhibit tumor growth and metastasis without having any effect on normal hematopoietic cells. Based on these findings, α₄β₁ integrin is a promising target for the treatment of both cancer and certain inflammatory diseases.

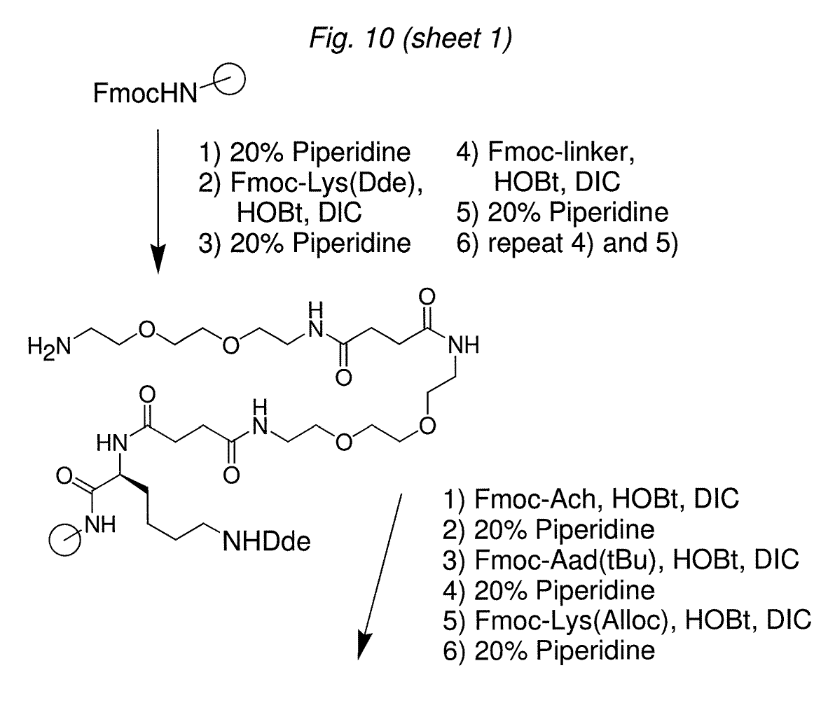

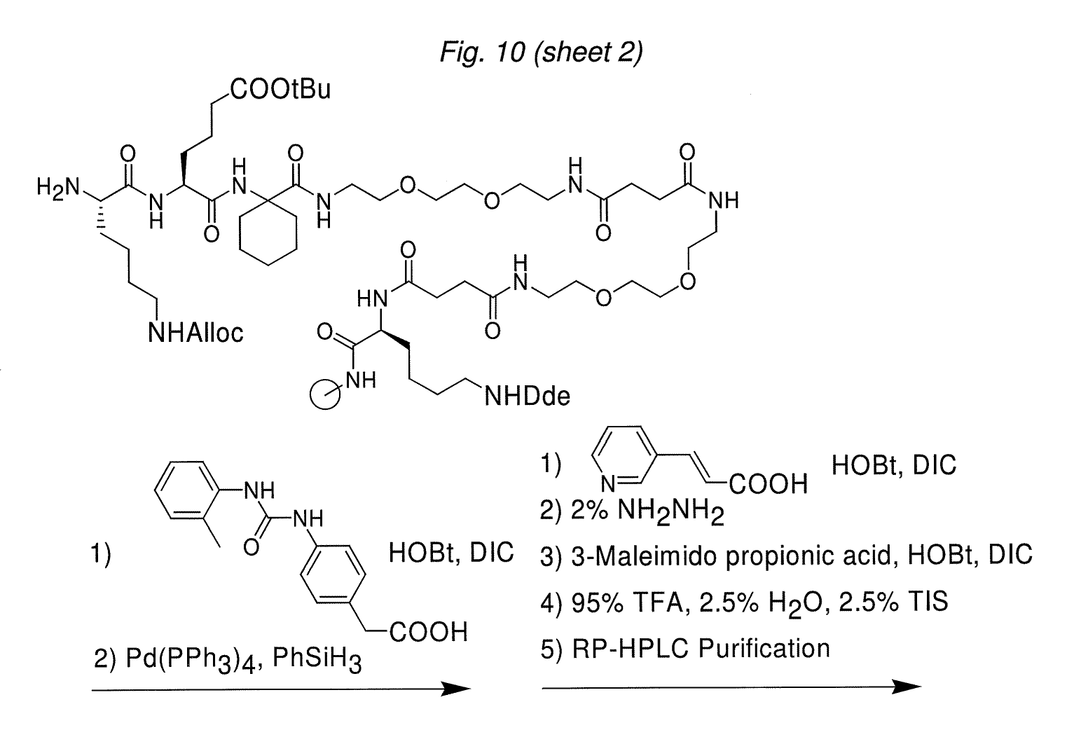

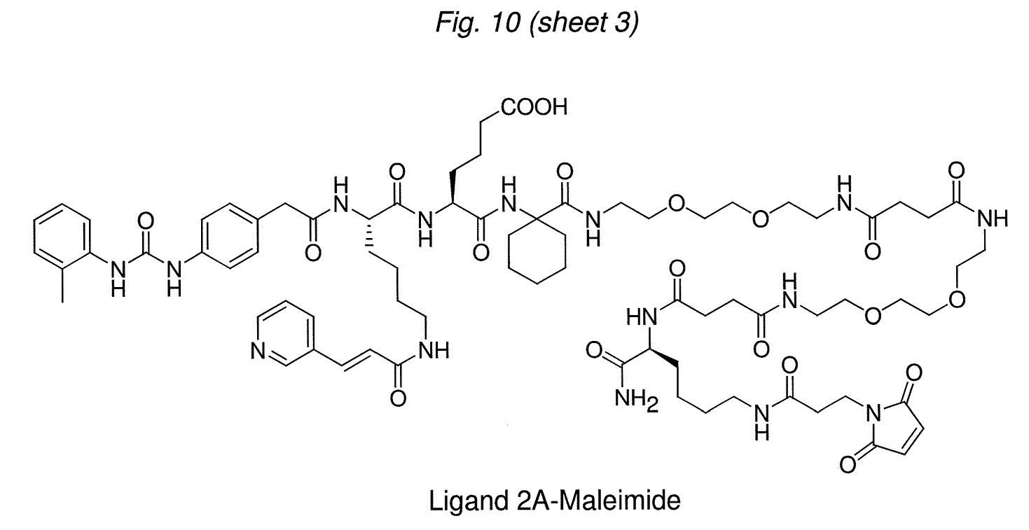

Chemical synthesis of Ligand 2A-Maleimide: Ligand 2A having a maleimide functional group was synthesized using synthetic FMOC protected chemistry. See, e.g., Hermanson, “Thiol-reactive chemical reactions” In Bioconjugate Techniques, Academia Press (1996). After synthesis, the Ligand 2A-Maleimide conjugate was purified using a C18 HPLC column. MALDI-TOF mass spectrometry revealed a mass of 1550 Da, which is close to the calculated mass. The structure and synthetic scheme of Ligand 2A-Maleimide is shown in FIG. 10.

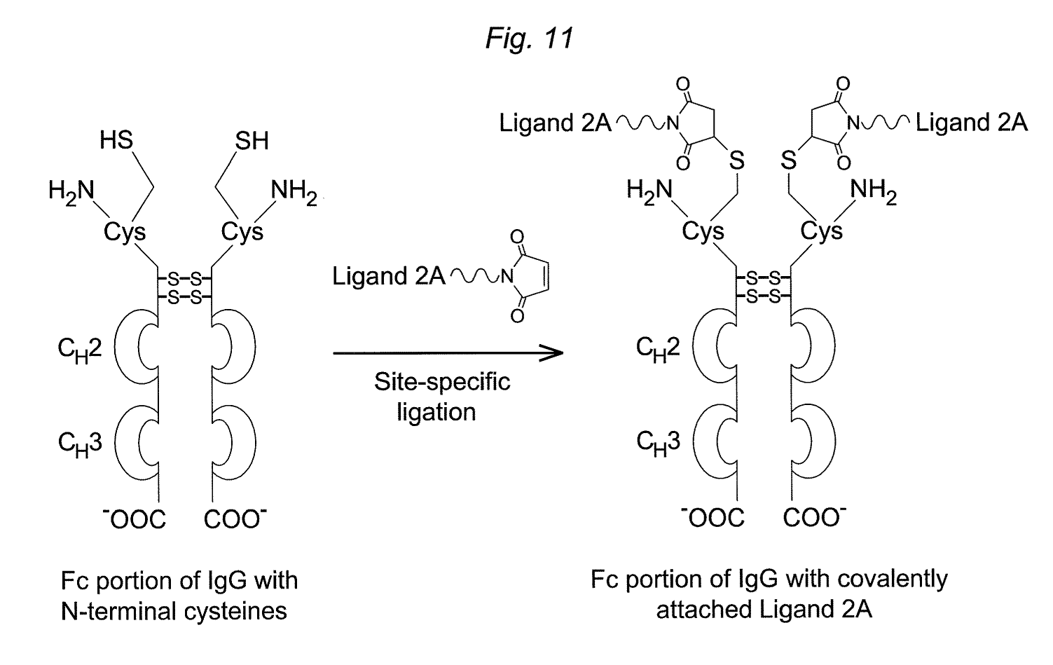

N-terminal site-specific conjugation of Ligand 2A-Maleimide: Malemide can react with thiol (i.e., sulfhydryl) functional groups and form a stable thioether bond. Maleimide reactions are specific for thiol groups in the pH range of 6.5-7.5. At pH 7.0, the reaction of maleimide with thiols proceeds at a rate 1000 times greater than its reaction with amines. This type of conjugation is beneficial to proteins which have few cysteines, such as antibodies. As such, modified IgG1 Fc fragments having N-terminal cysteine residues (i.e., hIgG1-Cys-Fc) can be used for conjugating Ligand 2A-Maleimide thereto. For example, 200 μg of hIgG1-Cys-Fc (4 nmoles) was mixed with 40 nmoles of Ligand 2A-Maleimide in a 100 μl volume of 0.1 M NaHPO₄, 150 mM NaCl₂, 10 mM EDTA, pH 7.2 and incubated for 4 h at room temperature. After conjugation, free Ligand 2A-Maleimide was removed using Sephadex G-50 spin columns. A schematic representation of the site-specific conjugation of Ligand 2A-Maleimide to hIgG1-Cys-Fc is shown in FIG. 11. The purified product was separated using 10% SDS-PAGE and Western blot analysis was performed as described above. The Ligand 2A-hIgG1-Cys-Fc conjugate displayed a higher molecular weight than hIgG1-Cys-Fc alone, indicating that Ligand 2A had been attached to hIgG1-Cys-Fc.

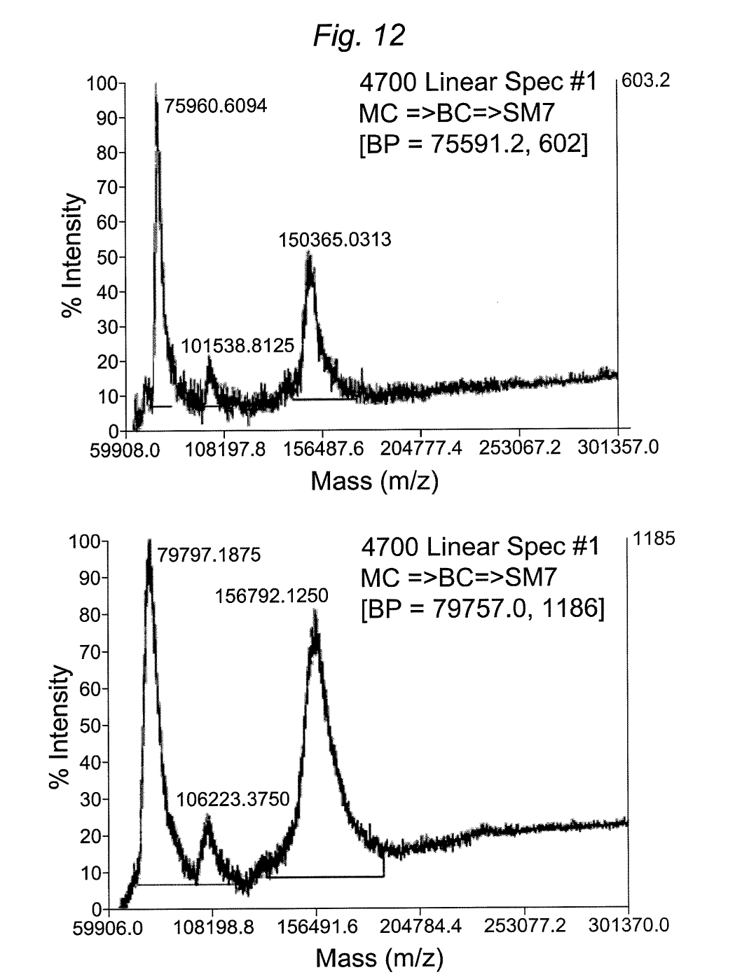

Non-site specific conjugation of Ligand 2A-Maleimide: Although many proteins and peptides do not naturally contain cysteine residues with free thiol groups, disulfide bonds can be reduced by reducing agents to generate free thiols. Alternatively, thiolating agents such as 2-iminothiolane (Traut's reagent) can be used to modify existing amino groups and introduce free thiol groups. For non-site specific conjugation of Ligand 2A-Maleimide to hIgG1, 10 mg of whole human IgG1 (Gamunex®, Talecris Biotherapeutics, Inc.; Research Triangle Park, N.C.) was purified by passing through a SephedexG-100 spin column and incubated with a 50 Molar excess of 2-iminothiolane (2-IT) in 200 μl of buffer (0.1 M NaHPO₄, 150 mM NaCl₂, 10 mM EDTA, pH 7.2) and incubated for 1 h at room temperature. The excess 2-IT was removed by passing thru SephadexG-50. A 50 Molar excess of Ligand 2A-Maleimide was added and incubated in a constant shaker overnight at 4° C. Unconjugated Ligand 2A-Maleimide was removed by passing thru SephadexG-50 spin columns and the eluate was collected. The number of Ligand 2A molecules attached to each antibody was determined. MALDI-TOF mass spectrometry revealed that the molecular weight of the human IgG1 was 150,365 Da (FIG. 12A), whereas the molecular weight of the Ligand 2A-hIgG1 conjugate was 156,972 Da (FIG. 12B). Since the molecular weight of Ligand 2A-Maleimide is 1550 Da, the mass spectrum indicated that around 4 Ligand 2A molecules were conjugated to every human IgG1 antibody molecule.

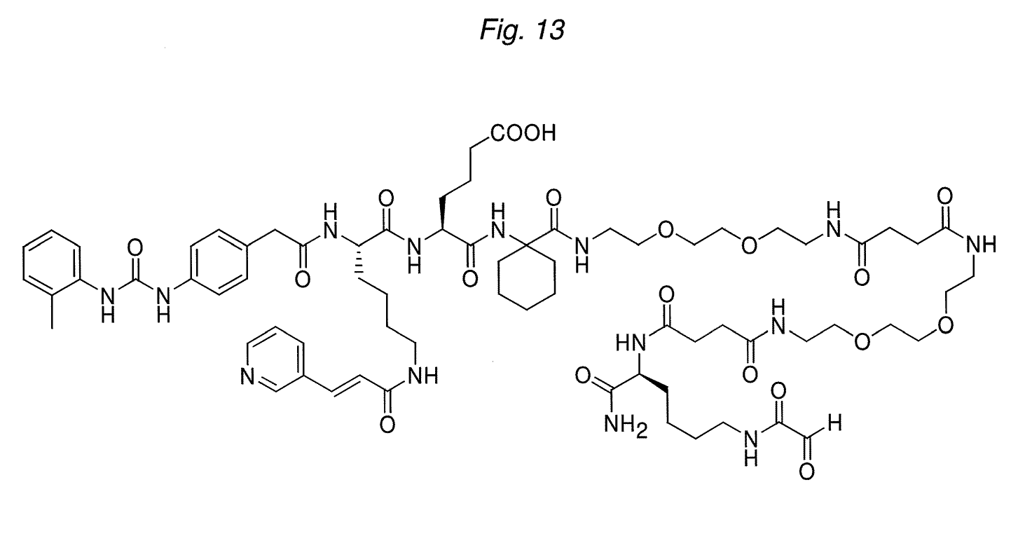

Chemical synthesis of Ligand 2A-Glyoxylyl: Ligand 2A-Glyoxylyl, which contains a hydrophilic linker and a glyoxylyl functional group, can also be synthesized using synthetic FMOC protected chemistry. See, e.g., Zhao et al., Bioconjugate Chemistry, 10:424-430 (1999). The structure of a Ligand 2A-Glyoxylyl conjugate is shown in FIG. 13. The mass spectrometry data for the Ligand 2A-Glyoxylyl conjugate was as follows: Calculated: 1454; Observed: 1455, 1456, 1457, 1458. Cysteine residues such as those present on the N-termini of the modified IgG1 Fc fragments described herein (i.e., hIgG1-Cys-Fc) can react very specifically with a glyoxylyl group at pH 7 or below, forming a relatively stable thioazolidine bridge. This specific reaction will not affect other cysteines present in the protein and can be performed efficiently at neutral pH to keep the protein in its native conformation. The Ligand 2A-hIgG1-Cys-Fc conjugate can then be purified using a Protein G column.

### Functional Properties of the Antibody Conjugates

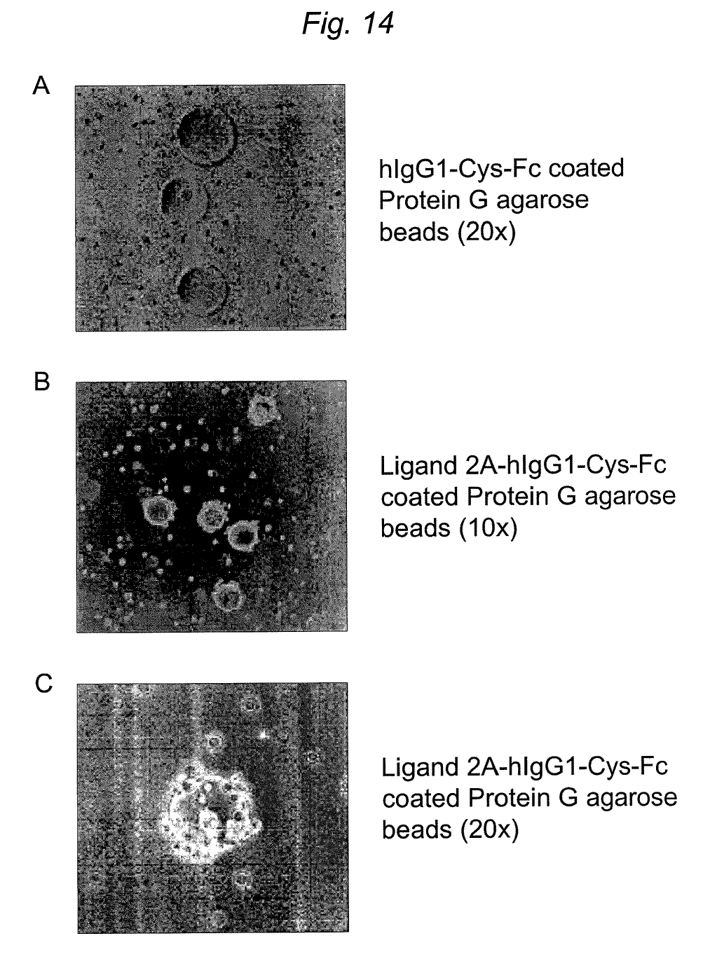

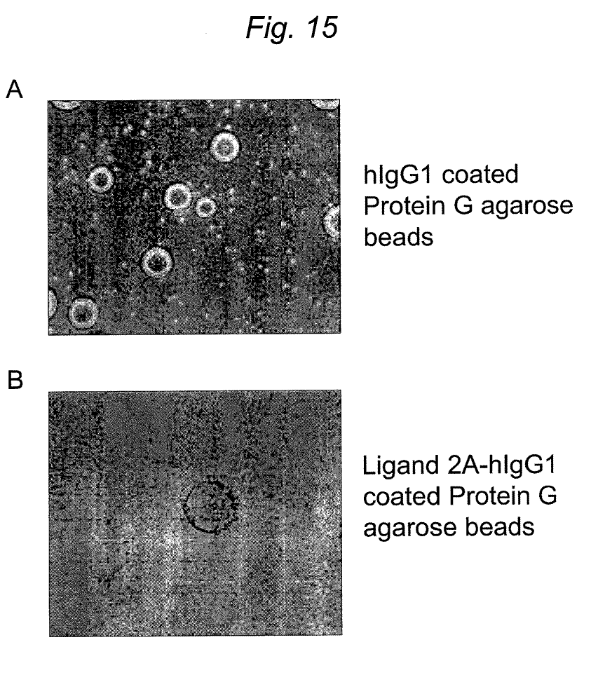

Cell binding studies on beads: Ligand 2A-hIgG1-Cys-Fc and unconjugated hIgG1-Cys-Fc were incubated with 10 μl of Protein A agarose beads (Bio-Rad Laboratories) for 3 h. In this manner, the whole bead was coated with either Ligand 2A-hIgG1-Cys-Fc or IgG1-Cys-Fc. Unbound Ligand 2A-hIgG1-Cys-Fc or IgG1-Cys-Fc was removed by washing the beads three times with phosphate buffered saline (PBS). The beads were incubated with 10% fetal bovine serum (FBS) containing RPMI medium (Invitrogen Corp.; Carlsbad, Calif.) for 1 h to avoid non-specific protein-protein interactions. 1 μl of Ligand 2A-hIgG1-Cys-Fc or IgG1-Cys-Fc coated beads was incubated with 100,000 MOLT-4 cells (a human T-cell lymphoma cell line which expresses α₄β₁ integrin) and monitored under the microscope every 10 min. As shown in FIG. 14, MOLT-4 cells bound to Ligand 2A-hIgG1-Cys-Fc coated beads, but not to IgG1-Cys-Fc coated beads. FIG. 15 shows that similar results were obtained with whole hIgG1 and Ligand 2A-hIgG1. As such, IgG molecules isolated from pooled human plasma can be used as a therapeutic agent against a particular target by conjugating a ligand specific for that target to the isolated IgG molecules.

Cell binding studies using flow cytometry: Additional cell targeting studies were performed with Ligand 2A-hIgG1-Cys-Fc and hIgG1-Cys-Fc. For example, K562 cells (a chronic myeloid leukemia cell line which expresses beta-1 integrin, but not the alpha-4 subunit) was used as a negative control and compared to K562 cells transfected with the alpha-4 subunit. K562 and K562 alpha-4-expressing cells were incubated either with 1 μg of Ligand 2A-hIgG1-Cys-Fc or hIgG1-Cys-Fc for 30 min on ice with occasional shaking. Cells were washed with PBS and FITC-conjugated goat anti-human IgG Fc-specific antibody was added (Rockland Immunochemicals, Inc.) at a 1:1000 dilution and incubated for 20 min. Cells were washed three times with PBS and analyzed using double color Beckman-Coulter flow cytometry, which revealed that K562 alpha-4-expressing cells showed a shift when stained with Ligand 2A-hIgG1-Cys-Fc, whereas hIgG1-Cys-Fc did not stain K562 alpha-4-expressing cells and Ligand 2A-hIgG1-Cys-Fc did not stain wild-type K562 cells. These results indicate that Ligand 2A-hIgG1-Cys-Fc selectively binds to the α₄β₁ integrin receptor on K562 alpha-4-expressing cells. Similar results were obtained using whole hIgG1 and Ligand 2A-hIgG1 and MOLT-4 cells, where 98% of the cells showed staining with Ligand 2A-hIgG1, but only 2% showed staining with hIgG1. Thus, Ligand 2A-hIgG1-Cys-Fc and Ligand 2A-hIgG1 specifically target α₄β₁ integrin-expressing cells.

Antibody-dependent cell-mediated cytotoxicity (ADCC): In the ADCC assay, natural killer (NK) cells are referred as “effector” cells and K562 and K562 alpha-4-expressing cells are referred as “target” cells. During cell killing, the cytosolic enzyme lactate dehydrogenase (LDH) is released in the supernatant such that estimating LDH levels in the supernatant provides an indication of the degree of cell cytotoxicity. 1×10⁴ target cells were plated in 96-well round-bottom plates with effector cells at various effector cell:target cell (EC:TC) ratios. Because NK cell susceptibility and expression of LDH varies from cell line to cell line, it is important to optimize the number of effector cells and target cells needed for the study. For these studies, effector cells at a 10:1 EC:TC ratio were used. Cell cytotoxicity was determined by estimating released LDH from the cell supernatant (Promega Corp.). All concentrations of test compounds described herein refer to final concentrations.

Extensive controls were used. For example, spontaneous lysis of target cells was determined using target cells in the presence of test compounds, but without effector cells. Similarly, spontaneous lysis of effector cells was determined using effector cells in the presence of test compounds, but without target cells. Blank correction was also used, which corresponded to media without cells. Maximum LDH release was defined as effector cells that were lysed with Triton-X45 minutes prior to the end of the incubation. A blank correction for maximum LDH release was used and an equal volume of Triton-X45 was added to these wells. 4 wells per treatment group were used. Effector cells were incubated with target cells and test compounds at 37° C. with 5% CO₂. After a 4 hour incubation period, cells were spun down at 850 rpm for 4 minutes. 50 μl of supernatant was collected from each well and added to 96-well flat-bottom plates. LDH release was assayed following the manufacturer's instructions using a CytoTox 96 non-radioactive cyotoxicity assay (Promega Corp.). Absorbance was measured at 490 nm. Percent cytotoxicity was then calculated using the following formula:

$$\% Lysis = \frac{\begin{matrix}Experimental - Effector spontaneous release - \\ Target spontaneous release\end{matrix}}{\begin{matrix}Target maximum release - \\ Target spontaneous release\end{matrix}} \times 100.$$

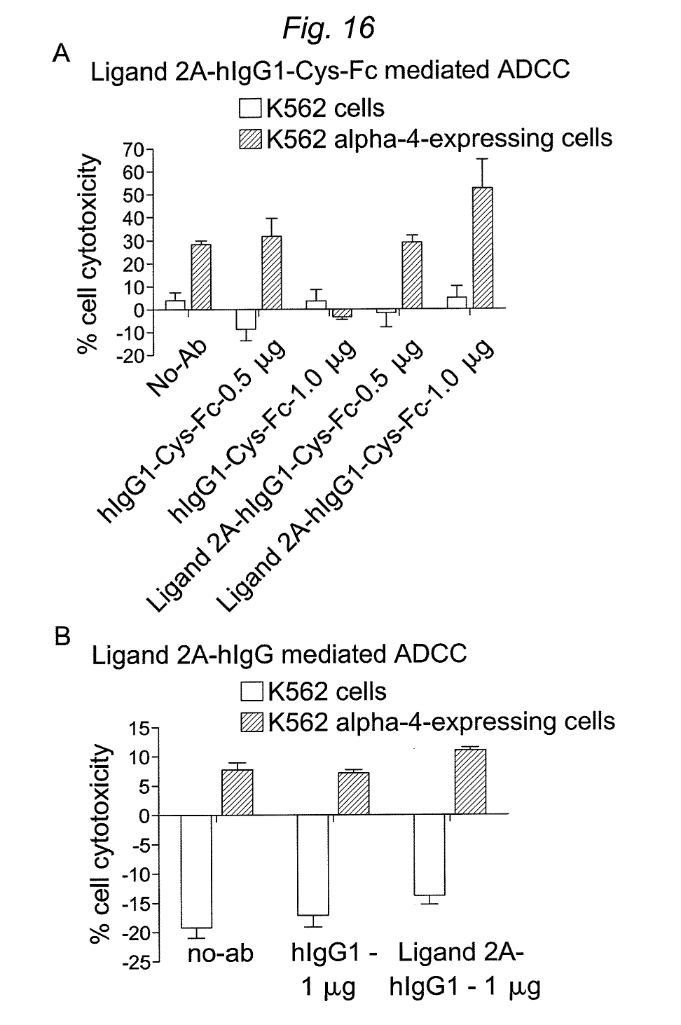

To study the effect of Ligand 2A-hIgG1-Cys-Fc on cell cytotoxicity, 0.5 μg or 1.0 μg of Ligand 2A-hIgG1-Cys-Fc or hIgG1-Cys-Fc were incubated with 10,000 target cells (K562 cells or K562 alpha-4-expressing cells) for 10 min and 100,000 NK cells (EC:TC ratio of 10:1) were added and incubated for 4 h at 37° C. with 5% CO₂. The percentage of cell killing was determined by the amount of LDH present in the cell supernatant. As shown in FIG. 16A, the percent of NK cell killing at 1.0 μg of Ligand 2A-hIgG1-Cys-Fc increased about 2-fold compared to 0.5 μg of Ligand 2A-hIgG1-Cys-Fc. The lack of a significant change in the killing of wild-type K562 cells indicates that the addition of Ligand 2A-hIgG1-Cys-Fc specifically kills those cells which express α4β₁ integrin. The addition of hIgG1-Cys-Fc to K562 cells or K562 alpha-4-expressing cells did not show any significant change in cell cytotoxicity. FIG. 16B shows that similar results were obtained when cell cytotoxicity experiments were performed with Ligand 2A-hIgG1 (whole IgG1 molecule) and hIgG1 at an EC:TC ratio of 1:1, which indicates that conjugation of ligands to whole IgG molecules can be useful in tumor cell killing.

This example demonstrates that: (1) using site-directed mutagenesis, N-terminal cysteine residues can be successfully introduced into the Fc fragment of human IgG1 or any other immunoglobulin isotype from humans or other species (e.g., dogs, rats, mice, etc.); (2) targeting agents can be successfully conjugated to whole human IgG1 or Fc fragments thereof using either site-specific or non-site specific conjugation techniques; (3) Ligand 2A or other ligands specific for targets associated with diseases or disorders such as cancer, infectious diseases, or neurological disorders can be synthesized with maleimide or glyoxylyl functional groups and attached to whole human IgG1 or Fc fragments thereof; (4) antibody conjugates comprising Ligand 2A attached to either Fc fragments or whole IgG1 molecules display target specificity in flow cytometric and cell binding assays and specifically kill cells that express the target receptors; and (5) cytotoxicity is mediated by recruiting effector cells (e.g., NK cells) through interactions between Fc receptors present on the effector cells and the Fc portion of the antibody conjugate.

### Example 3

### Synthesis of Antibody Conjugates Using Ketone-Oxime Conjugation

This example illustrates the synthesis of antibody conjugates of the present invention using a ketone-oxime conjugation method comprising derivatizing primary amine functional groups present on an antibody molecule with an N-succinimidyl levulinic acetate linker and reacting the methyl-ketone group generated on the antibody molecule with an aminoxy functional group present on a targeting agent to form an oxime bond. As such, the ketone-oxime conjugation method can be used to conjugate targeting ligands such as small organic molecules or peptides to the heavy chain constant region of antibodies or antibody fragments for imaging or therapeutic applications without interfering with their functions.

Solid Phase Synthesis of Ligand 2A with an Aminoxy Functional Group

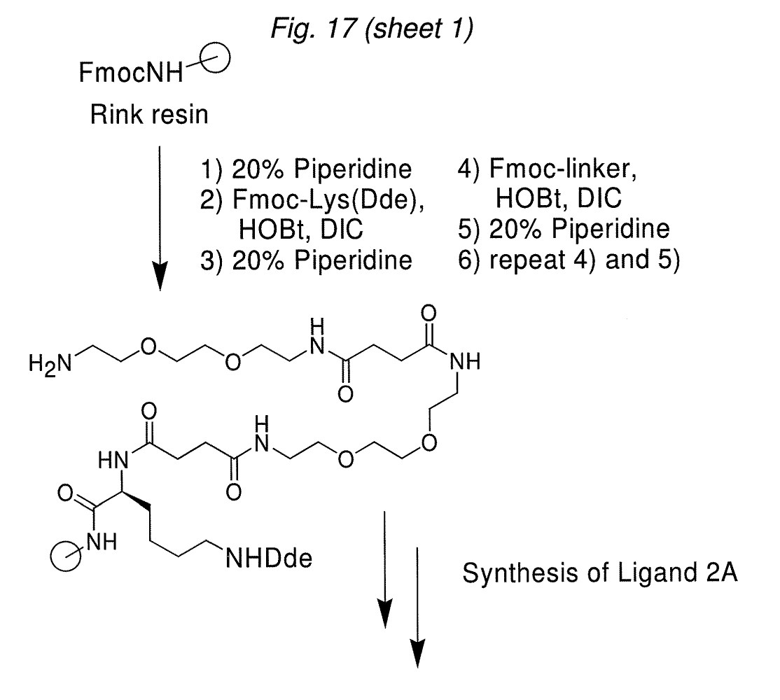

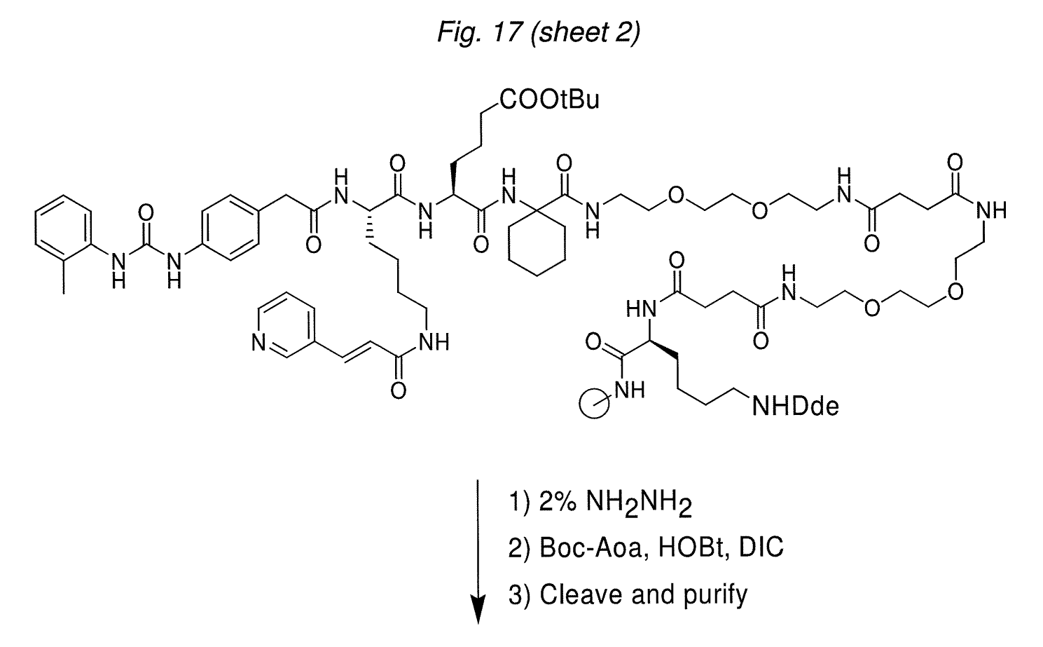

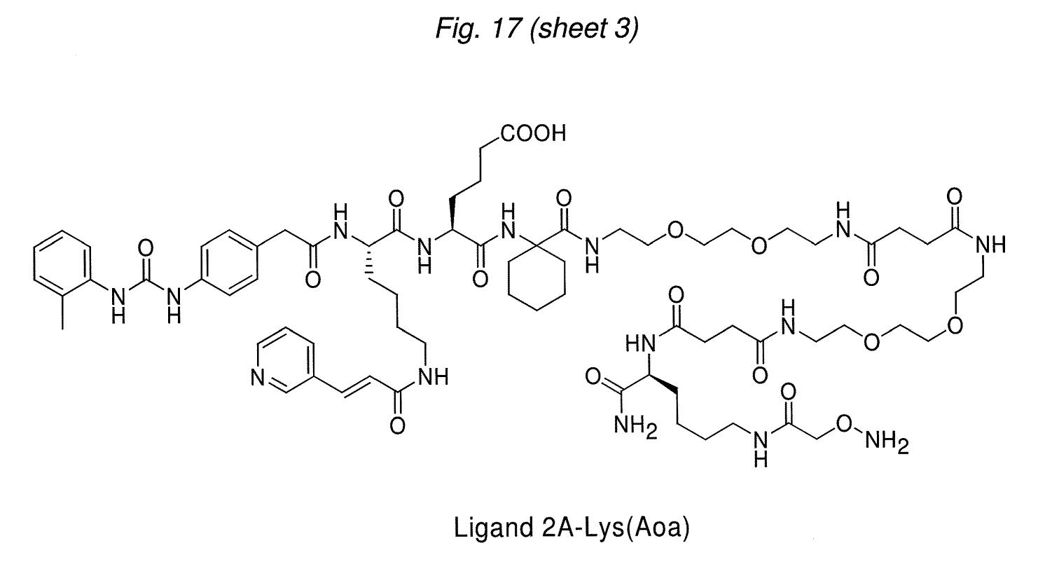

Ligand 2A (also known as LLP2A) was designed to have aminoxyacetic acid (Aoa) attached to the side-chain of Lys and two hydrophilic linkers between Ligand 2A and Lys(Aoa). The synthesis was performed on rink amide MBHA resin by a standard solid phase peptide synthesis approach using Fmoc-tBu chemistry and HOBt/DIC coupling. The synthetic scheme for Ligand 2A-Lys(Aoa) is shown in FIG. 17.

Fmoc-Lys(Dde)-OH was first coupled to the resin, followed by coupling of two linkers. Then, Ligand 2A-Lys(Aoa) was constructed as follows: Fmoc-Ach-OH (0.037 g, 0.0975 mmol) was dissolved in a solution of HOBt (0.015 g, 0.0975 mmol) and DIC (15 μL, 0.0975 mmol) in DMF, and was then added to the beads. The coupling was carried out at room temperature for 2 h. After filtration, the beads were washed with DMF (3×2 ml), MeOH (3×2 ml), and DMF (3×2 ml), respectively, three times each. The Fmoc deprotection group was removed with 20% piperidine twice (5 min, 15 min). After washing with DMF, MeOH, and DMF respectively, the beads were then subjected to additional coupling and deprotection cycles stepwise with Fmoc-Aad(tBu) and Fmoc-Lys(Alloc) in the same manner as described above. After removal of Fmoc, a solution of 2-(4-(3-o-tolylureido)phenyl)acetic acid (0.092 g, 0.325 mmol), HOBt (0.05 g, 0.325 mmol), and DIC (51 μL, 0.325 mmol) in DMF was added to the beads. The reaction was conducted at room temperature overnight. The beads were washed with DMF (3×12 ml), methanol (3×2 ml), and DMF (3×2 ml). The Alloc protecting group was removed with Pd(PPh3)4 (0.2 eq.), PhSiH3 (20 eq.) in DCM, 30 min, twice. After washing with DCM, DMF, MeOH, and DMF, a solution of trans-3-(3-pyridyl)acrylic acid (0.0485 g, 0.325 mmol), HOBt (0.05 g, 0.325 mmol), and DIC (51 μl, 0.325 mmol) in DMF (2 ml) was added to the beads. The coupling proceeded at room temperature overnight. A Kaiser test was negative. The beads were thoroughly washed with DMF, MeOH, and DMF. The Dde protecting group was removed with 2% NH₂NH₂ in DMF twice (5 min, 10 min). The beads were washed again with DMF, MeOH, and DMF as described above, followed by addition of a solution of Boc-Aoa (3 eq.), HOBt (3 eq.), and DIC (3 eq.). The coupling reaction was conducted at room temperature overnight. The beads were thoroughly washed with DMF, MeOH, and DCM and then dried under vacuum for 1 h before adding a cleavage mixture of 95% TFA: 2.5% water: 2.5% TIS. Cleavage of compounds from the resin and removal of side-chain protecting group were achieved simultaneously over 2 h at room temperature. The liquid was collected and concentrated. The dark blue crude products were precipitated with cold ether and purified by semipreparative reversed-phase HPLC (Vydac column, 20 mm×250 mm, 5 μm, 300 Å, C₁₈) with 45 min gradient from 100% aqueous media (0.1% TFA) to 100% CH₃CN (0.1% TFA). The flow rate was 5.0 ml/min. UV detection was carried out at 214 nm.

The purification was performed on a Beckman System Gold HPLC system (Fullerton, Calif.). The homogeneity of the compound was checked by analytical HPLC (Vydac column, 4.6 mm×250 mm, 5 μm, 300 Å, C₁₈), using 25 min gradient from 100% aqueous media (0.1% TFA) to 100% CH₃CN (0.1% TFA), at a flow rate of 1 ml/min and UV detection at 214, 220, 254, and 280 nm. The purity was determined to be >95%. The identity of the compounds was confirmed by Matrix-assisted laser desorption/ionization time of flight mass spectrometry (MALDI-TOF MS) on a Bruker BIFLEX III mass spectrometer (Billerica, MA). MALDI-TOF MS: 1472 (MH⁺).

### Covalent Attachment of Ligand 2A to Antibodies Using Ketone-Oxime Conjugation

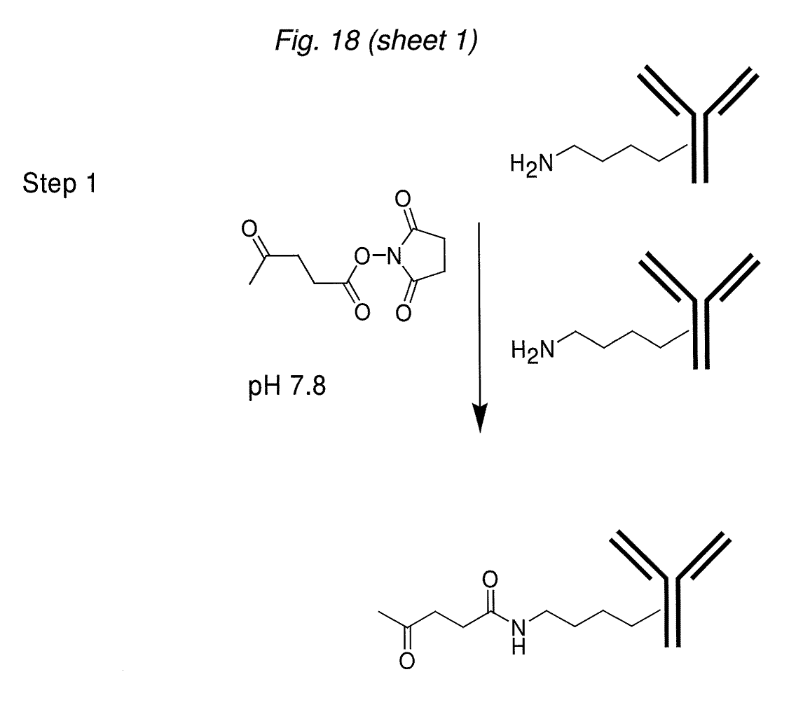

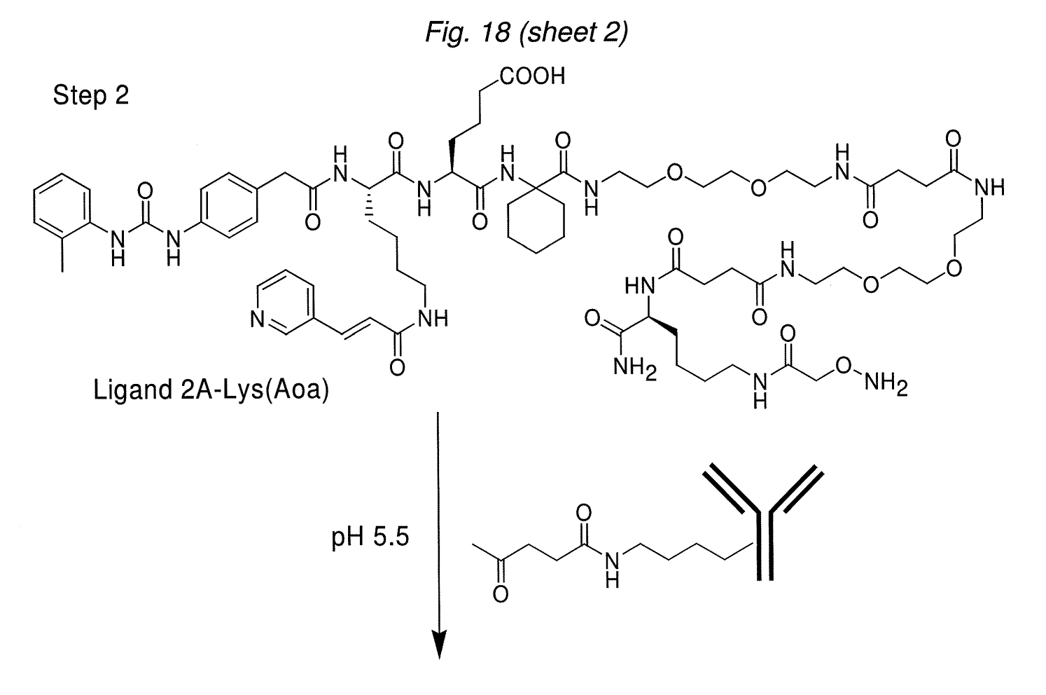

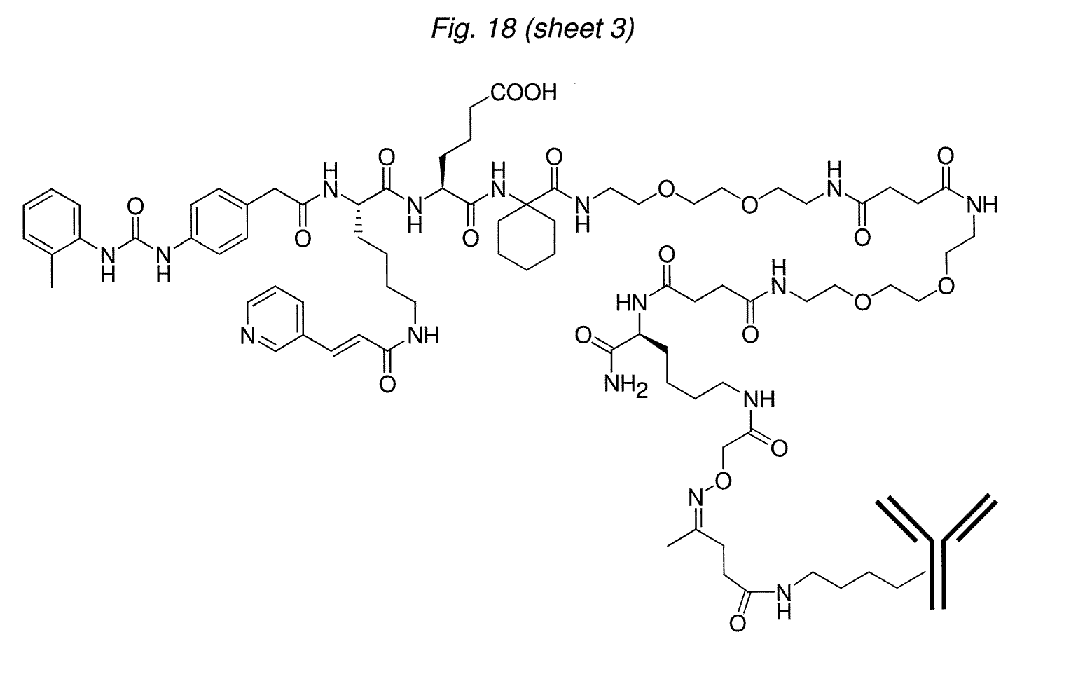

FIG. 18 illustrates a scheme using ketone-oxime conjugation to covalently attach a peptidomimetic targeting ligand such as Ligand 2A to the heavy chain constant region of a whole antibody molecule for immunotherapy or for imaging a tumor, organ, or tissue. The ketone-oxime conjugation method comprises two steps: (1) introduction of ketone functional groups onto primary amines (e.g., ε-amino groups of lysine residues) present on the antibody by acetylation of the heterobifunctional linker N-succinimidyl levulinic acetate; and (2) cross-linking aminoxy functional groups present on the targeting agent (e.g., Ligand 2A-Lys(Aoa)) with the ketone groups present on the antibody molecule to form a stable covalent ketone-oxime bond. With regard to the first step, a slightly alkaline pH of about 7.6-8.0 can be favorable and antibodies are generally stable at this pH. The second step is very specific and the rate of reaction can be more favorable at an acidic pH of about 5.5-6.5. Since antibodies are stable at this pH and the conjugation method takes a short amount of time (up to 4 h), ketone-oxime conjugation is particularly useful for covalently attaching targeting agents to antibodies or fragments thereof for immunotherapy and imaging applications.

Before conjugation, the antibody can be purified by passing it through a Sephedex-G100 molecular sieving column to remove impurities and to equilibrate it with the ketone linker conjugation buffer (e.g., 40 mM triethanolamine, pH 7.8). The conjugation can be performed in 50 mM phosphate buffer, pH 7.8. Ketone linker attachment to the antibody molecule can be performed with the antibody at different molar ratios (e.g., 10:1, 20:1, 30:1, etc.) for 1 h at room temperature. After conjugation, the excess free linker can be removed by passing the mixture through a Sephedex-G100 spin column. The targeting agent (e.g., Ligand 2A-Lys(Aoa)) can then be attached to the ketone linker-conjugated antibody at different molar ratios (e.g., 10:1, 20:1, 30:1, etc.) at room temperature for 4 h in 40 mM TEA, pH 5.5. Conjugation can be confirmed by determining the mass of the antibody before and after conjugation of the targeting agent using MALDI-TOF mass spectrometry.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A conjugate comprising:
(a) an antibody having at least a portion of a constant region; and
(b) a targeting agent comprising a functional group covalently attached to a complementary functional group present in said constant region.

2. A conjugate in accordance with claim 1, wherein said antibody is selected from the group consisting of IgG, IgM, IgA, IgD, IgE, and isotypes thereof.

3. A conjugate in accordance with claim 1, wherein said portion of said constant region comprises at least two heavy chain constant domains.

4. A conjugate in accordance with claim 3, wherein said portion of said constant region further comprises a hinge region.

5. A conjugate in accordance with claim 1, wherein said antibody comprises a dimer of heavy chain fragments with each heavy chain fragment independently comprising at least two heavy chain constant domains.

6. A conjugate in accordance with claim 5, wherein said dimer of heavy chain fragments comprises an Fc fragment.

7. A conjugate in accordance with claim 5, wherein each heavy chain fragment does not comprise a variable domain.

8. A conjugate in accordance with claim 1, wherein said targeting agent comprises a ligand.

9. A conjugate in accordance with claim 8, wherein said ligand comprises a peptide or peptidomimetic specific for an integrin receptor.

10. A conjugate in accordance with claim 9, wherein said integrin receptor is α₄β₁ integrin.

11. A conjugate in accordance with claim 10, wherein said peptidomimetic specific for α₄β₁ integrin has the formula:

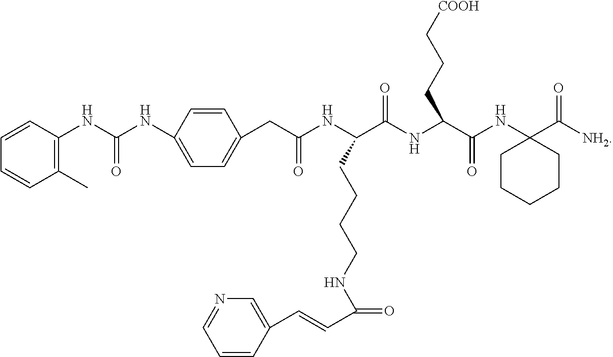

12. A conjugate in accordance with claim 8, wherein said ligand comprises a peptide or peptidomimetic specific for a lectin receptor, an immunoglobulin receptor, a protein aggregate, an infectious agent, a toxin, a metabolite, or a drug.

13. A conjugate in accordance with claim 1, wherein said functional group comprises a maleimide group.

14. A conjugate in accordance with claim 13, wherein said complementary functional group comprises a thiol group.

15. A conjugate in accordance with claim 1, wherein said functional group comprises an oxidized 3,4-dihydroxy-phenylalanine (DOPA) group.

16. A conjugate in accordance with claim 15, wherein said complementary functional group comprises an amine group.

17. A conjugate in accordance with claim 1, wherein said functional group comprises an aminoxy group.

18. A conjugate in accordance with claim 17, wherein said complementary functional group comprises a ketone group.

19. A conjugate in accordance with claim 18, wherein said ketone group is present on a linker attached to an amine group present in said constant region.

20. A conjugate in accordance with claim 1, wherein said complementary functional group is not present at the amino-terminus or carboxyl-terminus of said antibody.

21. A conjugate in accordance with claim 1, wherein said antibody further comprises a carboxyl-terminal polyarginine linker.

22. A conjugate in accordance with claim 21, wherein said polyarginine linker further comprises a therapeutic agent or an imaging agent attached thereto.

23. A conjugate in accordance with claim 22, wherein said therapeutic agent comprises a radionuclide or a toxin.

24. A conjugate in accordance with claim 22, wherein said imaging agent is selected from the group consisting of a radionuclide, biotin, a fluorophore, a fluorescent protein, horseradish peroxidase, and alkaline phosphatase.

25. A method for treating a disease or disorder in a subject in need thereof, said method comprising:
administering to said subject a therapeutically effective amount of a conjugate comprising an antibody having at least a portion of a constant region and a targeting agent comprising a functional group covalently attached to a complementary functional group present in said constant region.

26. A method in accordance with claim 25, wherein said disease or disorder is selected from the group consisting of cancer, an inflammatory disease, an autoimmune disease, an infectious disease, and a neurological disorder.

27. A method in accordance with claim 25, wherein said portion of said constant region comprises at least two heavy chain constant domains.

28. A method in accordance with claim 25, wherein said antibody comprises a dimer of heavy chain fragments with each heavy chain fragment independently comprising at least two heavy chain constant domains.

29. A method in accordance with claim 28, wherein said dimer of heavy chain fragments comprises an Fc fragment.

30. A method in accordance with claim 25, wherein said targeting agent comprises a ligand.

31. A method in accordance with claim 30, wherein said ligand comprises a peptide or peptidomimetic specific for an integrin receptor.

32. A method in accordance with claim 25, wherein said complementary functional group is not present at the amino-terminus or carboxyl-terminus of said antibody.

33. A method for imaging a tumor, organ, or tissue, said method comprising:
(a) administering to a subject in need of such imaging, a conjugate comprising an antibody having at least a portion of a constant region and a targeting agent comprising a functional group covalently attached to a complementary functional group present in said constant region; and
(b) detecting said conjugate to determine where said conjugate is concentrated in said subject.

34. A method in accordance with claim 33, wherein said portion of said constant region comprises at least two heavy chain constant domains.

35. A method in accordance with claim 33, wherein said antibody comprises a dimer of heavy chain fragments with each heavy chain fragment independently comprising at least two heavy chain constant domains.

36. A method in accordance with claim 35, wherein said dimer of heavy chain fragments comprises an Fc fragment.

37. A method in accordance with claim 33, wherein said targeting agent comprises a ligand.

38. A method in accordance with claim 37, wherein said ligand comprises a peptide or peptidomimetic specific for an integrin receptor.

39. A method in accordance with claim 33, wherein said antibody further comprises a carboxyl-terminal polyarginine linker.

40. A method in accordance with claim 39, wherein said polyarginine linker further comprises an imaging agent attached thereto.

41. A method in accordance with claim 40, wherein said imaging agent is selected from the group consisting of a radionuclide, biotin, a fluorophore, a fluorescent protein, horseradish peroxidase, and alkaline phosphatase.

42. A method in accordance with claim 41, wherein radiation from said radionuclide is used to determine where said conjugate is concentrated in said subject.

43. A method in accordance with claim 41, wherein fluorescence from said fluorophore or fluorescent protein is used to determine where said conjugate is concentrated in said subject.

44. A method in accordance with claim 33, wherein said complementary functional group is not present at the amino-terminus or carboxyl-terminus of said antibody.

45. A kit for immunotherapy comprising:
(a) a conjugate comprising an antibody having at least a portion of a constant region and a targeting agent comprising a functional group covalently attached to a complementary functional group present in said constant region; and
(b) directions for use of said conjugate in immunotherapy.

46. A kit for imaging a cell, tumor, organ, or tissue comprising:
(a) a conjugate comprising an antibody having at least a portion of a constant region and a targeting agent comprising a functional group covalently attached to a complementary functional group present in said constant region; and
(b) directions for use of said conjugate in imaging said cell, tumor, organ, or tissue.


SEQUENCE LISTING (4 sequences)

SEQ ID NO: 1 - DNA, 42 bp, Artificial Sequence
  site-directed mutagenesis forward primer Fc-cys-FP
cttgtcacgaattcgtgctcggccatggttagatctgtggag

SEQ ID NO: 2 - DNA, 42 bp, Artificial Sequence
  site-directed mutagenesis reverse primer Fc-cys-RP
ctccacagatctaaccatggccgagcacgaattcgtgacaag

SEQ ID NO: 3 - DNA, 60 bp, Artificial Sequence
  region of nucleotide sequence of pFUSE- hIgG1-Fc2 vector expressing human IgG1 Fc fragment used to mutate codon after signal sequence of heavy chain fragment to cysteine codon
ttgcactaagtcttgcacttgtcacgaattcgatatcggccatggttagatctgacaaaa

SEQ ID NO: 4 - DNA, 54 bp, Artificial Sequence
  nucleotide sequence of mutated clone hIgG1- Cys-Fc expressing modified form of human IgG1 Fc region having an N-termial cysteine in both heavy chain fragments
ttgcactaagtcttgcacttgtcacgaattcgtgctcggccatggttagatctg